(19) United States
(12) Patent Application Publication
(10) Pub. No.: US 2019/0167814 A1
(43) Pub. Date: Jun. 6, 2019

(54) Treatment And/Or Prevention Of DNA-Triplet Repeat Diseases Or Disorders

(71) Applicant: UNIVERSITÉ DE LAUSANNE, Lausanne (CH)

(72) Inventors: Vincent DION, Ecublens (CH); Lorene AESCHBACH, Chavannes-Pres-Renens (CH); Cinzia CINESI, Saint-Sulpice (CH)

(73) Assignee: UNIVERSITÉ DE LAUSANNE, Lausanne (CH)

(21) Appl. No.: 16/093,028

(22) PCT Filed: Apr. 13, 2017
(86) PCT No.: PCT/EP2017/058940

§ 371 Date: Oct. 11, 2018

(30) Foreign Application Priority Data
Apr. 14, 2016 (EP) 16165203.7

(51) Int. Cl. A61K 48/00; C12N 9/22; A61P 25/00; A61P 21/00

(52) U.S. Cl. CPC A61K 48/0066; C12N 9/22; A61K 48/0058; A61P 21/00; A61K 48/0091; A61P 25/00; A61K 48/0008

(57) ABSTRACT

The present invention refers to the field of DNA repair and to compositions, kits and methods for the treatment and/or prevention of DNA-triplet repeat diseases or disorders.

### FIELD OF THE INVENTION

The present invention refers to the field of DNA repair and to compositions, kits and methods for the treatment and/or prevention of DNA-triplet repeat diseases or disorders.

### BACKGROUND OF THE INVENTION

Repetitive DNA sequences are hotspots for genome instability because they pose a particular challenge to the DNA repair machinery. Their mutation often leads to disease. For example, tracts of CAG/CTG triplets (henceforth referred to as CAG repeats) reaching beyond a threshold of about 35 units cause at least 14 different cureless neurological and neuromuscular diseases¹. In addition, they become highly dynamic: their length changes at high frequencies in both somatic and germline cells throughout the lifetime of an individual.

The molecular mechanisms governing CAG repeat instability appears to revolve around their ability to fold into non-B-DNA structures when exposed as single-stranded DNA (ssDNA)¹. These unusual structures are mistaken for damaged DNA. The subsequent repair is error-prone due to the repetitive nature of the sequences and their structure-forming ability. Another non-mutually exclusive model is that DNA damage within the repeat tract triggers repair, which is, in turn, error-prone due to secondary structures formed by these sequences. In support for these models, several DNA repair pathways promote the instability of expanded CAG repeats, including mismatch repair (MMR), double-strand break (DSB) repair, transcription-coupled nucleotide excision repair (TC-NER), base excision repair (BER), as well as DNA replication¹. In contrast, single-strand break (SSB) repair and signaling via the DNA damage response (DDR) antagonize CAG repeat instability². Therefore, CAG repeats represent an opportunity to understand the interaction and interdependence of several different DNA repair pathways at naturally-occurring sequences.

Importantly, repeat length determines in large part the severity of the diseases caused by expanded repeats¹. It has therefore been proposed that contracting the repeat tract would be beneficial. Repeat expansions, on the other hand, would further exacerbate the disease symptoms.

Currently, there is no treatment that specifically shrinks CAG repeats. This is, in part, because the assays used to measure repeat instability are tedious, slow, and/or can only survey instability in one direction. Consequently, the understanding of the mechanism of CAG repeat instability remains poor.

### SUMMARY OF THE INVENTION

The present invention provides a kit for the treatment and/or prevention of a DNA-triplet repeat disease comprising a gene delivery vector, said vector comprising
i) a Cas9 nickase optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

A further object of the present invention is to provide a kit for the treatment and/or prevention of a DNA-triplet repeat disease comprising
i) a first gene delivery vector comprising a Cas9 nickase optimized for gene editing in mammalian cultured cell lines, embryonic stem (ES) cells, induced pluripotent stem cells (iPSCs), or in vivo, and
ii) a second gene delivery vector comprising at least one single guide RNA (sgRNA), or crRNA and tracrRNA, recognizing a target sequence comprising 16 to 25 nucleotide wherein said target sequence is present immediately upstream of a protospacer adjacent motif (PAM).

A further object of the present invention is to provide a gene delivery vector comprising
i) a Cas9 nickase optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

A further object of the present invention is to provide a gene delivery vector for use in the treatment and/or prevention of DNA-triplet repeat diseases, said vector comprising
i) a Cas9 nickase optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

A further object of the present invention is to provide pharmaceutical composition comprising
i) a vector comprising a Cas9 nickase optimized for gene editing in mammalian cultured cell lines, embryonic stem (ES) cells, induced pluripotent stem cells (iPSCs), or in vivo, and at least one single guide RNA (sgRNA), or crRNA and tracrRNA, recognizing a target sequence comprising 16 to 25 nucleotides wherein said target sequence is present immediately upstream of a protospacer adjacent motif (PAM), or
ii) a first gene delivery vector comprising an endonuclease Cas9 optimized for gene editing in mammalian cultured cell lines, embryonic stem (ES) cells, induced pluripotent stem cells (iPSCs), or in vivo, and a second gene delivery vector comprising at least one single guide RNA (sgRNA), or crRNA and tracrRNA, recognizing a target sequence comprising 16 to 25 nucleotide wherein said target sequence is present immediately upstream of a protospacer adjacent motif (PAM).

A further object of the present invention is to provide methods of treating and/or preventing DNA-triplet repeat diseases and uses of pharmaceutical compositions of the invention in the treatment and/or prevention of DNA-triplet repeat diseases.

### DESCRIPTION OF THE FIGURES

FIG. 1: DSBs within CAG repeats lead to expansions and contractions.

A) GFP-based assay to detect changes in repeat length. B) Representative flow cytometry profiles after expression of a ZFN in GFP(CAG)₁₀₁ using the protocol from³. C) Representative flow cytometry profiles with increased doxycycline (dox) induction time uncovering an increase in GFP⁻ cells on ZFN expression in GFP(CAG)₁₀₁ cells (arrow). D) Quantification of the ZFN experiments in (C) revealed that ZFN induces the appearance of GFP⁻ and GFP⁺ cells. ZFNs are composed of two different ZFN arms, each fused to a Fokl nuclease that must dimerize to be active. ZFN50 and ZFN51 are individual ZFN arms. The dashed line represents the number of cells present in gates set to include the dimmest (GFP⁻) or brightest (GFP⁺) 1% of the cells when a control vector, pCDNA3.1 Zeo, is transfected. Error bars are standard errors from 15 replicates for experiments with both ZFN arms, 12 for the single ZFN transfections. E) Quantification of GFP⁺ and GFP⁻ cells obtained after expression of the indicated vectors. Dashed line: dimmest (GFP⁻) or brightest (GFP⁺) 1% of the cells transfected with the Cas9 nuclease vector and the empty gRNA vector, pPN10. The error bars are s.e.m. Number of replicates per treatment: pcDNA+gDM1d, n=3; pcDNA+gCTG, n=5; Cas9 m4+ gCTG, n=4; Cas9+ gDM1d, n=3; Cas9+ gCTG, n=7. FC: flow cytometry; dox, doxycycline.

FIG. 2: The Cas9 nickase causes CAG repeat contraction.

A) Quantification of the effect of Cas9 nickase expression in GFP(CAG)₁₀₁ cells. Number of replicates per treatment: gCTG n=37; gCAG n=3; gDM1d n=3.

Quantification of gCTG experiments include also results from Cas9 nickase expression treated with DMSO and siRNAs against vimentin. Dashed line: dimmest (GFP⁻) or brightest (GFP⁺) 1% of the cells transfected with the Cas9 nickase vector and the empty gRNA plasmid. B) Quantification of GFP⁻ and GFP⁺ cells after 5 days (1 transfection) or 12 days (3 transfections) of Cas9 nickase expression with gCTG in GFP(CAG)₁₀₁ cells. Number of replicates per treatment: 5 days, n=37 (same as in A); 12 days, n=4 (*P=0.001, using a Wilcoxon U-test, compared with 5-day treatment). Dashed line: dimmest (GFP⁻) or brightest (GFP⁺) 1% of the cells transfected with the Cas9 nickase vector and the empty gRNA vector over the indicated period of time. C) SP-PCR (top) and its quantification (bottom) of DNA isolated from GFP(CAG)₁₀₁ cells after Cas9 nickase expression with or without gCTG (shown: 100 pg DNA per PCR) of pPN10 (50 pg DNA per PCR) for 12 days contained within the 25th to 75th percentile of GFP intensities (bulk), and GFP+ cells Cas9 nickase expression with gCTG or pPN10. b: no DNA blank. D) The ability of Cas9 nickase to induce contractions is repeat-length dependent. Dashed line: dimmest (GFP⁻) or brightest (GFP⁺) 1% of the cells transfected with the Cas9 nickase vector and the empty gRNA plasmid in the indicated cell line. For each cell line the 1% threshold is determined independently. The error bars are s.e.m. Number of replicates per treatment: GFP(CAG)₂₇₀, n=3; GFP(CAG)₁₀₁, n=37 (same as in A); GFP(CAG)₄₂, n=3; GFP(CAG)₁₈, n=2, GFP(CAG)₀, n=13.

FIG. 3: Single-strand break repair is not involved in Cas9-nickase-induced repeat instability.

A) Left: XRCC1 knock down (n=4) did not affect the GFP expression in GFP(CAG)₁₀₁ cells (P=0.7 for both GFP⁻ and GFP⁺ cells, usog a Wilcoxon U-test). Dashed line: dimmest (GFP⁻) or brightest (GFP⁺) 1% of the cells transfected with the Cas9 nickase vector; the empty gRNA plasmid; and the indicated siRNAs. Right: western blot showing knockdown efficiency by siRNA against XRCC1. B) Left: same as A, but cells treated with the PARP inhibitor Oliparib (n=4, P=0.5 for GFP⁻ cells and P=0.4 for GFP⁺ compared to DMSO treated-cells, using a Wilcoxon U-test). Dashed line: dimmest (GFP⁻) or brightest (GFP⁺) 1% of the cells transfected with the Cas9 nickase vector together with the empty gRNA plasmid, and treated with either DMSO or Oliparib. Right: PAR levels 30 minutes and 24 hours after treatment with 100 μg/ml of Zeocin. The error bars represent the standard error.

FIG. 4: Mechanism of Cas9-nickase-induced repeat instability.

A) Quantification of the GFP⁻ and GFP⁺ cells on treatment with DMSO (n=20, which includes the amount of DMSO for treatment with one or two inhibitors in GFP(CAG)₁₀₁ cells; these controls were from each other), an ATR inhibitor (VE-821, n=5), or an ATM inhibitor (KU60019, n=5), or both (n=3). Dashed line: dimmest (GFP⁻) or brightest (GFP⁺) 1% of the cells transfected with the Cas9 nickase vector together with the empty gRNA plasmid and treated with the DMSO or the indicated inhibitor. B) Quantification of GFP⁻ and GFP⁺ cells on knockdown with siRNAs (siVIM n=13; siMSH2 n=14; siXPA n=9; siMSH2+siXPA n=4). siVIM quantifications include results from knockdown of vimentin with both 10 and 20 nM of siRNAs as the results were indistinguishable from each other. Dashed line: dimmest (GFP⁻) or brightest (GFP⁺) 1% of the cells transfected with the Cas9 nickase vector; the empty gRNA plasmid; the indicated siRNAs. C) Quantification of the GFP⁻ and GFP⁺ cells on combinatorial knockdown of the indicated siRNAs and the ATR inhibitor VE-821 (siVIM+DMSO, n=6; siMSH2+ATRi, n=3; siXPA+ATRi, n=6). Dashed line: dimmest (GFP⁻) or brightest (GFP⁺) 1% of the cells transfected with the Cas9 nickase vector together with the empty gRNA plasmid, and treated with the indicated inhibitor or siRNA. The error bars are s.e.m. *: P≤0.05

FIG. 5: Model for Cas9-nickase-induced repeat contraction.

The pathway indicated in grey is proposed to be active only when ATR is inhibited.

FIG. 6: Characterization of the GFP reporter assay and GFP⁺ and GFP⁻ cells isolated from GFP(CAG)₁₀₁.

A) Profile of GFP intensity in three cell lines isolated by FACS after six months of culturing compared to the starting population of GFP(CAG)₁₀₁. The repeat length in each clone is marked above the flow cytometry profiles. B) Same as A, but in the presence of 2 μg/ml dox induction for 5 days. C) Repeat length for clones isolated from the GFP⁻ and GFP⁺ populations from GFP(CAG)₁₀₁ cells. The distribution of repeat lengths between GFP⁻ and GFP⁺ cells were significantly different (P=1×10⁻⁵). D) Schematic representation of clones from C with mutations in the flanking sequences. *: Three different clones were isolated with the same deletion, two with 78 repeats, one with 77. E) Same as C, but with clones cultured in the presence of dox for 6 months. The distribution of repeat lengths between GFP⁻ and GFP⁺ cells were significantly different (P=0.025). F) Schematic representation of the deletions found after 6 months of culturing in the presence of dox. G) Same as G, except that the cells were exposed to DMSO. The distribution of repeat lengths between GFP⁻ and GFP⁺ cells were significantly different (P=0.035). H) Same as F, but for clones cultured in DMSO. *: The 19 bp insertion is a direct repeat of the 19 bp immediately found before the insertion.

FIG. 7: Assay optimization, the effect of ZFN and Cas9 nuclease on GFP(CAG)o and analysis of GFP⁺ and GFP⁻ clones collected after ZFN treatment.

A) Example of data quantification. The GFP⁺ and GFP⁻ gates are set as the top or bottom 1% of the control population, in this case transfected with pCDNA3.1 Zeo.

The same gates are used to determine the proportion of cells from the treated population that falls within these set gates have changed expression (red). B) Flow cytometry profile of cells treated with dox for an increasing amount of time. C) One of 10 flow cytometry experiments of GFP(CAG)ₒ cells transfected with vectors expressing both ZFN arms or with a control vector (pCDNA3.1 Zeo). D) Repeat tract lengths in GFP⁺ and GFP⁻ clones after treatment of GFP(CAG)₁₀₁ cells with both ZFN arms. Dashed grey bars: repeat size in the starting population: 101 CAG repeats. The distribution of repeat lengths between GFP⁻ and GFP⁺ cells were significantly different (P=5×10⁻⁴). E) Schematic representation of clones with deletions in the sequences surrounding the CAG repeat. F) One of two flow cytometry experiments comparing cells expressing the Cas9 nuclease and the gCTG or transfected with an empty gRNA vector (pPN10). G and H) Representative flow cytometry profiles showing that the number of GFP⁺ cells increases after two more transfections over a total period of 12 days compared to our standard 5-days treatment.

FIG. 8: Cas9 nickase induces repeat instability with a bias towards contractions. A) Expression levels of the Cas9 nuclease and Cas9 nickase do not account for the different effects of these two enzymes on the number of GFP⁻ and GFP⁺ generated.). Dashed line: dimmest (GFP⁻) or brightest (GFP⁺) 1% of the cells transfected with the indicated amount of the Cas9 nuclease or nickase vector together with the empty gRNA plasmid. B) Western of Cas9 levels for the experiment presented in A. C) Flow cytometry data results from GFP(CAG)₁₀₁ cells transfected with the Cas9 nickase and with either pPN10 or gCTG-expressing vector showing that changing the laser intensity, and thus the apparent GFP expression, does not change the results of the quantifications. D) As in C but with GFP(CAG)₂₇₀. E) Size of repeat in clones isolated from GFP(CAG)₁₀₁ cells transfected with the gCTG and the Cas9-nickase expressing vectors. The distribution of repeat lengths between GFP⁻ and GFP⁺ cells were significantly different (P=2×10⁻⁴). F) Schematic of the rearrangements from in 3 GFP⁺ clones from E. *: This clone contained a complex rearrangement with the 36 bp insertion that includes a 10 bp insertion followed by two direct repeats of 13 bp corresponding to the last 13 bp prior to the insertion. G) Same as in E, but with cells transfected with the Cas9 nickase together with gCAG. H) Schematic of the clones from G that had changes in the sequences flanking the repeat. *: This clone had a 19 CAG repeat expansions downstream of a duplication that included the 40 bp immediately upstream of the repeat tract and 36 more CAGs.

FIG. 9: Effect of siRNA and inhibitor treatments on GFP(CAG)₀ cells and knockdown efficiency.

A) Representative flow cytometry plots from siRNA knockdown experiments (MSH2: n=6; XPA: n=6; XRCC1: n=4;). B) Representative flow cytometry results for inhibitor experiments (ATMi: n=5; ATRi: n=5; PARPi: n=4). C) Western blot showing knockdown efficiency by the MSH2 and XPA siRNAs.

FIG. 10: The saCas9 nickase and nmCas9 nickase activity in GFP(CAG)₁₀₁ cells.

Quantification of the effect of sa/nm Cas9 nickases expression in GFP(CAG)₁₀₁ cells after 5 days. Dashed line: dimmest (GFP⁻) or brightest (GFP⁺) 1% of the cells transfected with the vector expressing the indicated Cas9 nickase orthologue and the corresponding empty gRNA plasmid. Number of replicates per treatment: saCas9 nickase+gCAG, n=2; saCas9 nickase+gAGC, n=2; saCas9 nickase+gGCA, n=3; saCas9+gCTG, n=2; saCas9+gTGC, n=2; saCas9 nickase+gGCT=3; nmCas9 nickase+gCAG, n=2; nmCas9 nickase+gAGC, n=2; nmCas9 nickase+gGCA, n=3; nmCas9+gCTG, n=2; nmCas9+gTGC, n=2; nmCas9 nickase+gGCT=3.

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise” and “comprising” also encompass the more restricted ones “consist” and “consisting”, respectively.

As used in the specification and claims, the singular form “a”, “an” and “the” include plural references unless the context clearly dictates otherwise.

As used herein, “at least one” means “one or more”, “two or more”, “three or more”, etc.

As used herein the terms “subject”/“subject in need thereof”, or “patient”/“patient in need thereof” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other aspects, the subject can be a normal subject.

The term does not denote a particular age or sex. Thus, adult and newborn subjects, whether male or female, are intended to be covered.

The terms “nucleic acid”, “polynucleotide,” and “oligonucleotide” are used interchangeably and refer to any kind of deoxyribonucleotide (e.g. DNA, cDNA, . . . )

or ribonucleotide (e.g. RNA, mRNA, . . . ) polymer or a combination of deoxyribonucleotide and ribonucleotide (e.g. DNA/RNA) polymer, in linear or circular conformation, and in either single—or double—stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity; i.e., an analogue of A will base-pair with T.

The term “vector”, as used herein, refers to a viral vector or to a nucleic acid (DNA or RNA) molecule such as a plasmid or other vehicle, which contains one or more heterologous nucleic acid sequence(s) (such as nucleic acid sequence(s) encoding the sgRNA, TRACR and CrRNA, CAS9 nickase, and is designed for transfer between different host cells. The terms “expression vector”, “gene delivery vector” and “gene therapy vector” refer to any vector that is effective to incorporate and express one or more nucleic acid(s), in a cell, preferably under the regulation of a promoter. A cloning or expression vector may comprise additional elements, for example, regulatory and/or post-transcriptional regulatory elements in addition to a promoter.

Any suitable vector can be employed that is effective for introduction of one or more nucleic acid(s) into cells. Preferably, the gene delivery vector of the invention is a viral vector, such as a lenti- or baculo- or preferably adeno-viral/adeno-associated viral (AAV) vectors, but other means of delivery or vehicles are known (such as yeast systems, microvesicles, gene guns/means of attaching vectors to gold nanoparticles) and are provided, in some embodiments, one or more of the viral or plasmid vectors may be delivered via liposomes, nanoparticles, exosomes, microvesicles, or a gene-gun. Most preferably, the gene delivery vector is selected from the group comprising an adeno-associated virus (AAV) and a lentivirus. Lentivirus of 1st, 2nd, and 3rd generation are described in Naldini et al, 2016 the contents of which is incorporated by reference.

Preferably, the lentivirus of the invention will be a lentivirus of third generation as described in Dull T et al., 1998, the contents of which is incorporated by reference.

The type of AAV surface protein determines the target tissue. Preferably, adeno-associated virus (AAV) will be selected from the group comprising AAV6 and AAV9, due to their broad tissue specificity and expression levels. AAV9 particularly has a minimal inflammatory response, thereby reducing the side effects. The viral particles will usually be administered by injection into the bloodstream. AAV6 can also be used with cultured patient-derived cells. This will be useful when using the approach in iPSCs or ES cells as described in the present disclosure.

Repetitive DNA sequences, such as DNA-triplet repeat, are hotspots for genome instability because they pose a particular challenge to the DNA repair machinery. Their mutation often leads to disease. For example, the expansion of CAG/CTG repeats causes at least 14 different DNA-triplet repeat diseases or disorders (Table 1) that all remain without a cure. They vary in prevalence from 1 in 8000 for myotonic dystrophy (DM1) to less than 1 in 100 000 for some spinocerebellar ataxias (SCAs). Each disease is clinically distinct, despite sharing the same mutation type, albeit in different genes. These diseases are all multisystemic with the skeletal muscles, heart, and the central nervous system (CNS) being particularly affected. Other tissues, however, can also be affected. This is the case for DM1 patients who tend to develop diabetes due to pancreatic deficiencies, as well as cataracts. In addition, each disease has a particular set of affected neurons. For example, in Huntington disease, the first neurons to degenerate are the striatal medium spiny neurons, whereas the cerebellar Purkinje cells are most affected in SCAs.

Surprisingly, the inventors of the present invention have shown that the CRISPR-Cas9 system may be implemented for the treatment and/or prevention of DNA-triplet repeat diseases or disorders.

Preferably, the DNA-triplet repeat disease or disorder of the invention is an expanded CAG/CTG triplet repeat disease, most preferably a neurological or neuromuscular disease. More preferably, the phenotype of the expanded CAG/CTG triplet repeat neurological or neuromuscular disease is reversible or essentially reversible, i.e. when the CAG/CTG triplet repeat is contracted the disease is cured or essentially cured clinical. Even more preferably, the CAG/CTG triplet repeat neurological or neuromuscular disease is selected from the non-limiting group comprising Dentatorubral-pallidoluysian atrophy, Fuchs' endothelial corneal dystrophy, Huntington disease, Huntington disease-Like 2, Myotonic Dystrophy type 1, Spinal and bulbar muscular atrophy, spinocerebellar ataxia 1, spinocerebellar ataxia 2, spinocerebellar ataxia 3, spinocerebellar ataxia 6, spinocerebellar ataxia 7, spinocerebellar ataxia 8, spinocerebellar ataxia 12, and spinocerebellar ataxia 17, which are listed in the below Table 1.

TABLE 1
Human neurological and neuromuscular disorders caused
by the expansion of expanded CAG repeats.

| | | Orientation of |
|---|---|---|
| Disease | Gene affected | the repeat tract |
| DRPLA | ATN1 | CAG |
| FECD | TCF4 | CTG |
| HD | HTT | CAG |
| HDL2 | JPH3 | CAG |
| DM1 | DMPK | CTG |
| SBMA | Androgen receptor | CAG |
| SCA1 | ATXN1 | CAG |
| SCA2 | ATXN2 | CAG |
| SCA3 | ATXN3 | CAG |
| SCA6 | CACNAIA | CAG |
| SCA7 | AtTXN7 | CAG |
| SCA8 | ATXN8 | CTG |
| SCA12 | PPP2R2B | CAG |
| SCA17 | TBP | CAG |

DRPLA: Dentatorubral-pallidoluysian atrophy; FECD: Fuchs' endothelial corneal dystrophy; HD: Huntington disease; HDL2: Huntington disease-Like 2; DM1: Myotonic Dystrophy type I; SBMA: Spinal and bulbar muscular atrophy; SCA: spinocerebellar ataxia.

Treatments that are currently being considered aim to alleviate the symptoms of the DNA-triplet repeat diseases rather than targeting their root cause. This means that an eventual treatment for one disease would not be efficacious for another disease. On the other hand, treatments that would target the cause of the disease, the expanded CAG/CTG repeats, would work for every disease, regardless of the clinical symptoms.

Several studies suggest that the length of the expanded repeat determines much of the severity of the disease with longer repeats leading to an earlier age of onset and worse symptoms. This has led to the hypothesis that shrinking (referred to as contracting) the repeat tract would effectively remove the underlying cause of the disease¹. That is, provided that the disease symptoms are reversible and that a treatment able to contract specifically the repeat tract are available.

It is becoming apparent that at least some of the expanded trinucleotide repeat disorders are reversible. For example, an inducible mouse model of DM1 could be rescued by shutting off the expression of a GFP reporter containing the 3′UTR of the DMPK gene, where an expanded repeat is located in DM1 patients⁴. Similarly, Zu et al⁵ used a mouse model for SCA1 with a transgene containing the cDNA of ataxin1 containing 82 CAGs driven by a tetracyclin-inducible promoter. They demonstrated that shutting off the expression of the transgene leads to a reversal of the molecular and physiological phenotypes of SCA1. Both of these examples worked even if the disease stage was well beyond the disease onset. Together, these two studies suggest that it is possible to reverse the disease symptoms even after a diagnostic has been made.

Repeat expansion is ongoing in somatic tissues throughout the disease progression. The accumulation of longer and longer repeat tracts over time is thought to precipitate disease progression. Indeed, preventing repeat instability in mouse models for Huntington disease slowed down the progression of the disease.

As used herein, “cas9” or “cas9 endonuclease” refers to “CRISPR-associated endonuclease 9” which is a bacterial RNA-directed nuclease that can be adapted for gene editing in mammalian cultured cell lines, embryonic stem (ES) cells, induced pluripotent stem cells (iPSCs), and even in vivo⁶. It works by inducing a double-strand break (DSB) to DNA that can be repaired in an error-prone manner by non-homologous end joining, or by supplementing with a homologous template containing the modifications to be made to the genome. In this latter case, homologous recombination is used to insert the modification.

Generally, Cas9 uses a single guide RNA (sgRNA) or a TRACR and CrRNA that recognize a target sequence composed of 16 to 25 nucleotides (e.g. 17, 18, 19, 20, 21, 22, 23, or 24 nucleotides), depending on which species the Cas9 comes from. The rest of the RNA is a scaffold whose sequence is also specific to the bacterial species from which the Cas9 enzyme comes from. In addition, Cas9 needs a Protospacer Adjacent Motif (PAM) sequence immediately after the target sequence for full efficiency.

Within the context of this disclosure, the Cas9 endonuclease of the invention is a nickase. Modified versions of the Cas9 endonuclease containing a single inactive catalytic domain, either RuvC- or HNH-, are called “nickases”. With only one active nuclease domain, the Cas9 nickase cuts only one strand of the target DNA, creating a single-strand break or “nick”, i.e. there is no deletion of the flanking region in contrast to cas9 endonuclease. Similar to the inactive dCas9 (RuvC- and HNH-), a Cas9 nickase is still able to bind DNA based on gRNA specificity, though nickases will only cut one of the DNA strands. Most preferably, the Cas9 nickase is optimized for gene editing in mammalian cultured cell lines, embryonic stem (ES) cells, induced pluripotent stem cells (iPSCs), or in vivo.

A Cas9 nickase small enough to be packaged into a gene delivery vector such as e.g. an AAV, and that can recognize a CAG repeat is selected from the non-limiting group comprising the Staphylococcus aureus Cas9 nickase, Neisseria meningitidis (NmeCas9) nickase, Parvibaculum lavamentivorans Cas9 nickase, Campylobacter lari Cas9 nickase Streptococcus pyogenes (Sp)Cas9 nickase and Campylobacter jejuni Cas9 nickase.

Examples of most preferred Cas9 nickases comprise the NmeCas9 nickase and the SpCas9 nickase. The NmeCas9 nickase is 1082 amino-acid long, and can recognize the sequence NNNNGNTG⁸. This nickase has been obtained by mutating the aspartic acid of the Cas9 endonuclease at position 16, to an alanine (D16A).

The SpCas9 nickase is a 1368 amino acid variant whose only targeting limitation is the requirement of a PAM consisting of NGG nucleotides immediately 3′ to the target site. This nickase has been obtained by mutating the aspartic acid of the Cas9 endonuclease at position 10, to an alanine (D10A).

The target nucleic acid sequence of the sgRNAs is usually CAG CAG CAG CAG CAG CAG CAG (SEQ ID No. 1) or CTG CTG CTG CTG CTG CTG CTG (SEQ ID No. 2), or any fragment thereof, which comprises at least two CAG or CTG repetitions respectively.

The full sequence of the sgRNA will be therefore selected from the group comprising GCAGCAGCAGCAGCAGCAGCAGGTTGTAGCTCCCTTTCTCATTTCGGAAACGA AATGAGAACCGTTGCTACAATAAGGCCGTCTGAAAAGATGTGCCGCAACGCTCT GCCCCTT (SEQ ID No. 3) and GCTGCTGCTGCTGCTGCTGCTGGTTGTAGCTCCCTTTCTCATTTCGGAAACGAA ATGAGAACCGTTGCTACAATAAGGCCGTCTGAAAAGATGTGCCGCAACGCTCT GCCCCTT (SEQ ID No. 4), or any fragment or variant of said sequences.

By variant is meant that the different species of Cas9 have different guide RNA scaffold differences and thus this sequence will change depending of the orthologue use or whether it has been optimized for expression efficiency. The target sequences (CAG or CTG repeats) will not change. In some cases it will be AGC AGC . . . or GCA GCA.

As discussed herein, Cas9 needs a Protospacer Adjacent Motif (PAM) sequence immediately after the target sequence for full efficiency. The PAM is a nucleotide motif that is recognized by the implemented Cas9 endonuclease and is different depending on the species of origin. The PAM sequence is often degenerate. For instance, the best studied Cas9, from Staphylococcus pyogenes Cas9 (SpyCas9), recognizes NGG, where N is any residue. However, it also recognizes NAG and NTG at lower frequencies⁷. This degeneration in the PAM as well as in the recognition sequence results in off-target recognition and mutations by Cas9 enzymes⁷.

Within the context of this disclosure, where a target sequence is present immediately upstream of a protospacer adjacent motif (PAM), it refers to the fact that the target sequence is flanked or followed, preferably at its 3′ end, by a PAM suitable for the Cas9.

Preferably, the Cas9 nickase is optimized for gene editing in mammalian cultured cell lines, embryonic stem (ES) cells, induced pluripotent stem cells (iPSCs), or in vivo. Examples of optimizations comprise the addition of a mammalian—such as human—codon or of a nuclear localization sequence-flanked wild-type, or both, to the Cas9 nickase sequence, or the addition of tag or changing the bacterial DNA sequence for codon optimization in mammalian species. In addition, it is possible to provide two halves of the Cas9 nickase tagged to intern separately for splicing of the complete enzyme in cells.

Preferable approaches to reduce off-target effects include the use of two sgRNAs that recognize closely spaced sequences on opposite strands together with Cas9 mutants that only nick the DNA⁶, and mutating the Cas9 protein either through molecular evolution or by mutating residues that stabilize the interaction of Cas9 with its target gene. Only one sgRNA is used at a time for in vivo gene editing since using both will induce DSBs, which we have shown promote expansions as well as contractions.

In an aspect of the invention, viral vectors are used as gene delivery vectors to deliver the complexes into a cell. Use of viral vectors as delivery vectors are known in the art. See for example U.S. Pub. 2009/0017543 the contents of which is incorporated by reference. Preferably, the gene delivery vector is a viral vector selected from the group comprising an adeno-associated virus (AAV) or a lentivirus.

Preferably a single vector system in which a single vector expresses both the sgRNA and the NmeCas9 nickase is used. An alternative approach that may generate higher viral titers is to use two different vector system: one expressing the sgRNA, the other the NmeCas9 Nickase cDNA.

Any suitable promoter or enhancer may be used that results in expression of one or more nucleic acid(s) into cells. Preferably, the expression of the sgRNA will be driven by a promoter preferably positioned upstream, e.g. contiguous to and upstream, such H1 or a U6 promoter, of the sequence encoding said sgRNA. Other tissue-specific promoters can be envisioned, such as the CMV promoter especially in cases where skeletal muscles are targeted.

Alternatively, the CamKII promoter appears especially suitable for expression in the CNS.

In one aspect, the promoter is an inducible promoter that can be turned on or off at certain stages of development of an organism or in a particular tissue. Preferably, the inducible promoter will be selected from the group comprising promoters whose activity is modified in response to heavy-metal ions, isopropyl-ß-D-thiogalactoside, hormones, progesterone antagonists or antibiotics. Most preferably, the inducible promoter will be selected from the group comprising Tetracycline or doxycycline (dox)-inducible promoter.

Preferable mammalian cultured cell lines, embryonic stem (ES) cells, induced pluripotent stem cells (iPSCs) will be selected—or will be derived from—the group comprising neurons, glia, satellite muscle cells, heart cells, hepatocytes, and fibroblasts.

The present invention also concerns a kit for the treatment and/or prevention of a DNA-triplet repeat disease comprising a gene delivery vector, said vector comprising
i) an endonuclease Cas9 optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

Usually, the target sequence comprising 16 to 25 nucleotides is selected from the group comprising CAG CAG CAG CAG CAG CAG CAG and CTG CTG CTG CTG CTG CTG CTG.

The kit for the treatment and/or prevention of a DNA-triplet repeat disease of comprises a gene delivery vector, which is selected from the group comprising an adeno-associated virus (AAV) and a lentivirus.

The invention further contemplates a kit for the treatment and/or prevention of a DNA-triplet repeat disease comprising
i) a first gene delivery vector comprising an endonuclease Cas9 optimized for gene editing in mammalian cultured cell lines, embryonic stem (ES) cells, induced pluripotent stem cells (iPSCs), or in vivo, and
ii) a second gene delivery vector comprising at least one single guide RNA (sgRNA), or crRNA and tracrRNA, recognizing a target sequence comprising 16 to 25 nucleotide wherein said target sequence is present immediately upstream of a protospacer adjacent motif (PAM).

The kits of the invention may also comprise a container and a label or package insert on or associated with the container. Suitable containers include, for example, bottles, vials, syringes, etc. The containers may be formed from a variety of materials such as glass or plastic. The container holds a composition which is effective for treating the disease of disorder of the invention and may have a sterile access port (for example the container may be an intravenous solution bag or a vial having a stopper pierceable by a hypodermic injection needle). Alternatively, or additionally, the kits may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The label or package insert may comprise instructions for use thereof. Instructions included may be affixed to packaging material or may be included as a package insert. While the instructions are typically written or printed materials they are not limited to such. Any medium capable of storing such instructions and communicating them to an end user is contemplated by this disclosure.

Also contemplated is a gene delivery vector comprising
i) an endonuclease Cas9 optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

The present invention also provides a gene delivery vector for use in the treatment and/or prevention of a DNA-triplet repeat disease, said vector comprising

i) an endonuclease Cas9 optimized for gene editing in mammalian cultured cell lines, embryonic stem (ES) cells, induced pluripotent stem cells (iPSCs), or in vivo, and

ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

The at least one sgRNA molecule or crRNA and tracrRNA molecules, the gene delivery vectors and the cell (single cell or population of cells) according to the invention can be formulated and administered to treat and/or prevent DNA-triplet repeat disease states by any means that produces contact of the sgRNA molecule or crRNA and tracrRNA molecules, the gene delivery vectors and the cell with its site of action in the patient in need thereof.

A typical pharmaceutical composition of the invention comprises i) a vector comprising an endonuclease Cas9 optimized for gene editing in mammalian cultured cell lines, embryonic stem (ES) cells, induced pluripotent stem cells (iPSCs), or in vivo, and at least one single guide RNA (sgRNA), or crRNA and tracrRNA, recognizing a target sequence comprising 16 to 25 nucleotides wherein said target sequence is present immediately upstream of a protospacer adjacent motif (PAM),

or ii) a first gene delivery vector comprising an endonuclease Cas9 optimized for gene editing in mammalian cultured cell lines, embryonic stem (ES) cells, induced pluripotent stem cells (iPSCs), or in vivo, and a second gene delivery vector comprising at least one single guide RNA (sgRNA), or crRNA and tracrRNA, recognizing a target sequence comprising 16 to 25 nucleotide wherein said target sequence is present immediately upstream of a protospacer adjacent motif (PAM).

Such pharmaceutical compositions of the invention further comprise one or more pharmaceutically acceptable carrier(s) or excipient(s) that are well known to the skilled in the art.

The term “carrier” refers to a diluent, adjuvant, or vehicle with which the active principle is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Pharmaceutically acceptable excipients include starch, glucose, lactose, sucrose, sodium stearate, glycerol monostearate, talc, sodium chloride, dried skim milk, glycerol, propylene glycol, water, ethanol and the like.

The pharmaceutical compositions may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include macrocrystalline cellulose, carboxymethyf cellulose sodium, polysorbate 80, phenyletbyl alcohol, chiorobutanol, potassium sorbate, sorbic acid, sulfur dioxide, propyl gallate, the parabens, ethyl vanillin, glycerin, phenol, parachlorophenol, gelatin, albumin and a combination thereof A thorough discussion of pharmaceutically acceptable excipients is available in REMINGTON'S PHARMACEUTICAL SCIENCES (Mack Pub. Co., N.J. 1991) which is incorporated by reference herein.

The invention also contemplates a method of treating and/or preventing a DNA-triplet repeat disease comprising administering a pharmaceutical composition of the invention to a subject in need thereof.

The present invention further contemplates a method of treating and/or preventing a DNA-triplet repeat disease comprising modifying, a target sequence comprising 16 to 25 nucleotides of interest in a single cell or a population of cells, and reintroducing the modified single cell or population of cells into the patient in need thereof.

Preferably, a biopsy or other tissue or biological fluid sample comprising the single cell or the population of cells may be necessary. Stem cells such as ES cells or pluripotent stem cell that can be generated directly from adult cells, such as iPSCs, are particularly preferred in this regard. Usually, the cell is selected—or derived—from the group comprising neurons, glia, satellite muscle cells, heart cells, hepatocytes, and fibroblasts.

Alternatively, the population of cells can be, e.g. an embryo.

The gene delivery vector or the one or more nucleic acid(s) encoding the sgRNA and/or Cas9 nickase can be introduced to the single cell or the population of cells via one or more methods known in the art. These one or more methods include, without limitation, microinjection, electroporation, calcium phosphate-mediated transfection, cationic transfection, liposome transfection, dendrimer transfection, heat shock transfection, nucleofection transfection, magnetofection, lipofection, optical transfection, proprietary agent-enhanced uptake of nucleic acids, and delivery via liposomes, immunoliposomes, virosomes, or artificial virions. For example, a plasmid containing a single cassette expressing the Cas9 nickase can be co-transfected with the sgRNA as PCR amplicons (Ran et al., 2013).

It will be appreciated that in the present method the modification following the introduction of the gene delivery vector, or the one or more nucleic acid(s) encoding the sgRNA (or crRNA and tracrRNA) and/or Cas9 nickase, to the single cell or the population of cells may occur ex vivo or in vitro, for instance in a cell culture and in some instances not in vivo. The sgRNA, or crRNA and tracrRNA, directs Cas9 nickase to and hybridizes to a target motif of the target sequence, thereby cleaving the target sequence.

In other aspects, it may occur in vivo. In case the population of cells is an embryo, then the gene delivery vector is introduced into said embryo by microinjection, in vivo. The gene delivery vector may be microinjected into the nucleus or the cytoplasm of the embryo.

Alternatively, the one or more nucleic acid(s) encoding the sgRNA and/or Cas9 nickase can also be delivered in the form of RNA.

To enhance expression and reduce possible toxicity, the one or more nucleic acid(s) encoding the sgRNA and/or Cas9 nickase, in the form of RNA, can be modified to include one or more modified nucleoside e.g. using pseudo-U or 5-Methyl-C.

Optionally, the one or more nucleic acid(s) encoding the sgRNA and/or Cas9 nickase can be under the regulation of regulatory elements in addition to a promoter.

By modification or alteration of a target sequence comprising 16 to 25 nucleotides of interest, it is meant inducing a nick on the genomic DNA of the single cell or the population of cells that contracts the CAG/CTG triplet repeat tract.

The modified single cell or population of cells is/are then reintroduced into the patient in need thereof by any route of administration and/or delivery methods known in the art as described below.

The compositions of the present invention may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For human use, the composition may be administered as a suitably acceptable formulation in accordance with normal human practice. The skilled artisan will readily determine the dosing regimen and route of administration that is most appropriate for a particular patient. The compositions of the invention may be administered by traditional syringes, needleless injection devices, “microprojectile bombardment gone guns”, or other physical methods such as electroporation (“EP”), “hydrodynamic method”, or ultrasound.

The compositions of the present invention may also be delivered to the patient, by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The compositions may be injected intra veniously or locally injected in the brain or muscle or electroporated in the tissue of interest such as muscle, brain, liver, heart, kidney(s), and hematopoietic system.

The present invention also contemplates one or more nucleic acid(s) encoding the sgRNA (or crRNA and tracrRNA) and/or the Cas9 nickase, as well as the plasmid containing the necessary regulatory elements.

Referring in more details to the Examples, our results show that the Cas9 nickase can be used to induce site-specific DNA gaps.

### EXAMPLES

### Example 1

Materials and Methods

Cell Culture

The GFP(CAG)₀ and GFP(CAG)₁₀₁ cells lines were a kind gift from John H. Wilson³. The cells tested negative for mycoplasma using the MycoAlert detection kit (Lonza) at the start of our experiments. The GFP(CAG)₁₅, GFP(CAG)₁₈, GFP(CAG)₄₂, GFP(CAG)₅₀, and GFP(CAG)₂₇₀ were isolated from populations grown for 6 months unperturbed or after transfection with the ZFN. They did not contain mutations in the region flanking the repeat tract. The cells were maintained at 37° C. with 5% CO₂ in Dulbecco's modified Eagle's medium (DMEM) glutamax, supplemented with 10% Fetal Bovine Serum (FBS), 100 U/mL penicillin (pen), 100 μg/mL streptomycin (strep), 15 μg/mL blasticidine and 150 μg/mL hygromycin. When the cells were destined for flow cytometry, they were kept in DMEM glutamax, with 10% of dialyzed calf serum, along with pen-strep. During the long term culturing, the cells were split 1 to 5 twice a week and the medium was supplemented with blasticidine and hygromycin to ensure continued expression of the TetR and GFP transgenes.

Plasmids and siRNA transfections, pharmacological inhibitors

The plasmids used in this study are found in Table 2. cDNA transfections were performed using 6×10⁵ cells/well in 12-well plates using a total of 1 μg of DNA and Lipofectamine 2000 (Life Technologies) per well. The culture medium was replaced 6 hours after transfection and 2 μg/mL of dox, diluted in DMSO, was added. Controls without dox were treated with DMSO alone. Forty-eight hours later, the medium was replaced and dox was freshly added. Flow cytometry, protein extraction, and/or DNA extraction were performed after another 48 hours of incubation.

TABLE 2
Plasmids

| Name | Content | Source |
|---|---|---|
| pCDNA3.1 | Empty vector | Life Technologies |
| Zeo | | |
| pcDNA3.3- | Cas9 D10A | via Addgene |
| TOPO - | | |
| Cas9_D10A | | |
| pCDNA3.3- | human Cas9 | via Addgene |
| TOPO | | |
| hCas9 | | |
| pPN10 | Empty gRNA | This study |
| pPN10- | pPN10 with (CAG)₆ gRNA - | This study |
| gCAG | PAM: CAG | |
| pPN10- | pPN10 with (CTG)₆ gRNA - | This study |
| gCTG | PAM: CTG | |
| pPN10- | pPN10 against a sequence in | This study |
| gDMd | the 3′ UTR of the DMPK gene | |
| pZFN50 | Single ZFN arm: 50 | Ref. 3 |
| pZFN51 | Single ZFN arm: 51 | Ref. 3 |

The siRNAs used in this study are found in Table 3. When transfecting with both a cDNA and a siRNA, 8×10⁵ cells/well were used along with 1 μg of DNA and 20 nM of siRNAs using Lipofectamine 2000. The medium was replaced 6 hours later and dox was added. Forty-eight hours after the first transfection, we performed a second siRNA transfection with RNAiMax (Life technologies) using half of the cells present and 20 nM of siRNA. We collected the cells to assess knockdown efficiency or GFP fluorescence analysis 48 hours later. When transfecting two siRNAs, we used a final siRNA concentration of 40 nM, where 20 nM of each individual siRNA were used. We found that single knockdowns at 20 nM were no different from those also containing 20 nM of the vimentin siRNA and were pooled for the statistical analyses and in the presented figures.

TABLE 3
siRNAs

| siRNA | Target | Sequence | SEQ IDs |
|---|---|---|---|
| siRNA-0001 | Vimentin | GAAUGGUACAAAU | SEQ ID |
| | | CCAAGU | No. 5 |
| siRNA-0002 | MSH2 | UCUGCAGAGUGUU | SEQ ID |
| | | GUGCUU | No. 6 |
| siRNA-0003 | XPA | GCUACUGGAGGCA | SEQ ID |
| | | UGGCUA | No. 7 |
| siRNA-0062 | XRCC1 | CAGUUUGUGAUCA | SEQ ID |
| | | CAGCACAGGAAU | No. 8 |

When using small molecule inhibitors (Table 4), the cells were treated as above, except that 2×10⁵ fewer cells/well were used. The medium, along with the dox and the inhibitors, were replaced for another 48 hours of treatment. Cell cycle analysis was performed after 96 hours of treatment. Briefly, the cells were fixed with 100% ethanol and treated with RNAseA (50 μg/mL) before adding propidium iodine (50 μg/mL). Flow cytometry analysis was performed as described below.

TABLE 4
Inhibitors

| Name inhibitor | Target | Concentration |
|---|---|---|
| Oliparib | PARP1/2 | 1 μM |
| KU60019 | ATM | 1 μM |
| VE-821 | ATR | 1 μM |

Flow cytometer and Cell Sorting

In preparation for flow cytometry analysis, cells were re-suspended in phosphate buffered saline (PBS) with 1 mM EDTA to a concentration of about 10⁶ cells/mL. For each condition, we measured at least 2×10⁵ events using a LSRII from BD. Data analysis was done using Flowing II. FACS was performed using a FACS Aria II (BD) or MoFlo Astrios (Beckman Coulter). For single clone analyses, we re-suspended the cells to a concentration of 2×10⁶ cells/mL and sorted the GFP⁻ and GFP⁺ cells. The cells were then expanded in DMEM glutamax supplemented with pen-strep, blasticidine, hygromycin, 5% FBS, and 5% dialyzed calf serum. For DNA isolation, cells were re-suspended to a concentration of 1.4×10⁷ cells/mL and we isolated between 4×10⁴ and 106 cells from the GFP− and GFP⁺ populations. We also isolated cells with GFP intensities between the 25th and 75th percentiles. For viability tests, cells were treated as described above except that 96 hours after the first transfection they were collected in PBS with 1 mM EDTA and 1 μM of TO-PRO-3 was added as a dead cell marker.

Quantification of GFP⁺ and GFP⁻ cells

To quantify the fold increase in the number of GFP⁺ or GFP⁻ cells, we first established gates that contained the top or bottom 1% of GFP expressing cells in the control treatment, for example the nickase plasmid transfected together with an empty gRNA vector (pPN10). For each treatment or cell line, therefore, the top and bottom 1% were adjusted to take any shift in GFP expression into account (e.g., due to the size of the repeat tract or the transfection protocol). In some cases, we adjusted the voltage of the flow cytometer laser to accommodate samples with very high or very low GFP expression. This adjustment did not interfere with the quantification (FIG. 8DE). Once the GFP gates established, we calculated the percentage of cells from the treated population (e.g., expressing both the Cas9 nickase and the gCTG) falling within these same gates. In cases where inhibitors or siRNAs were used, the control population expressed the Cas9 nickase, pPN10, and the inhibitor or siRNA. The 1% cut offs were used to keep a balance between having enough cells for robust statistics and the range of GFP expression in cells with a relatively homogeneous repeat length (for example see FIG. 6AB).

Repeat length determination and small pool PCR

To determine the repeat length of each sorted clone, we isolated DNA using the PeqGold MicroSpin Tissue DNA kit (PeqLab). The DNA was then amplified with primers 0437 and 0459 (Table 5). Several PCR reactions were set up with MangoTaq and the products were gel-extracted, pooled and sent for sequencing with the same primers used for the amplification. The repeat size was determined from at least two different amplification and sequencing reactions. The longest repeat size determined was used in the rare cases where the repeat length was not identical between the runs. Small-pool PCR was done as described¹³, except that primers 0459 and 0460 were used for the amplification. The probe was derived from a PCR product amplified with the same primers from a plasmid containing 40 repeats. The primers used to amplify the off-target loci are found in Table 5.

TABLE 5
Primers

| Primer | Locus | Sequence | SEQ IDs |
|---|---|---|---|
| 0437 | Pem1 intron | TACCAGGACA | SEQ ID No. 9 |
| | in the GFP | GCAGTGGTCA | |
| | cassette | | |
| 0459 | Pem1 intron | AAGAGCTTCCC | SEQ ID No. 10 |
| | in the GFP | TTTACACAACG | |
| | cassette | | |
| 0460 | Pem1 intron | TCTGCAAATT | SEQ ID No. 11 |
| | in the GFP | CAGTGATGC | |
| | cassette | | |
| 1251 | DMPK | GAGCGTGGGT | SEQ ID No. 12 |
| | | CTCCGCCCAG | |
| 1252 | DMPK | CACTTTGCGA | SEQ ID No. 13 |
| | | ACCAACGATA | |
| 1255 | ATN1 | ACTCAGCCTT | SEQ ID No. 14 |
| | | CTCTCCCATC | |
| 1256 | ATN1 | TGTAGGACAC | SEQ ID No. 15 |
| | | CTGGCTGTGA | |
| 1257 | AR | TAGGGCTGGG | SEQ ID No. 16 |
| | | AAGGGTCTAC | |
| 1258 | AR | CTCTGGGACG | SEQ ID No. 17 |
| | | CAACCTCTCT | |
| 1259 | ATXN1 | TTCCAGTTCA | SEQ ID No. 18 |
| | | TTGGGTCCTC | |
| 1260 | ATXN1 | GTGTGTGGGA | SEQ ID No. 19 |
| | | TCATCGTCTG | |
| 1269 | TBP | TTCTCCTTGC | SEQ ID No. 20 |
| | | TTTCCACAGG | |
| 1270 | TBP | GGGGAGGGAT | SEQ ID No. 21 |
| | | ACAGTGGAGT | |
| 1273 | PPP2R2B | GCAGCAAAGA | SEQ ID No. 22 |
| | | GCAGCCGCAG | |
| 1274 | PPP2R2B | CTGGTCCCAC | SEQ ID No. 23 |
| | | GGGAGGGCGG | |

Antibodies and western Blotting

Protein extraction was done using RIPA buffer and proteinase inhibitor cocktail tablets (Roche, Germany) and at least 10 μg of proteins were loaded onto a 6% or 10% Tris/glycine SDS polyacrylamide gels and transferred onto nitrocellulose membranes. The antibodies used in this study are found in Table 6. An Odyssey Infrared Imager (Licor) was used for signal detection.

TABLE 6
Antibodies

| Antibody | Species | Dilution | Source | Reference |
|---|---|---|---|---|
| Anti-Actin | Rabbit | 1:2000 | Sigma-Aldrich | A2066-.2ML |
| Anti-CRISPR- | Rabbit | 1:1000 | Abcam | ab204448 |
| Cas9 | | | | |
| Anti-MSH2 | Mouse | 1:2000 | Abcam | ab52266 |
| [3A2B8C] | | | | |
| Anti-PAR | Mouse | 1:1000 | Amsbio | 4335-AMC-050 |
| Anti-XPA [5F12] | Mouse | 1:2000 | Abnova | MAB6747 |
| Anti-XRCC1 | Mouse | 1:1000 | Abcam | ab1838 |
| [33-2-5] | | | | |

Statistics

When determining whether there were differences in the frequency of GFP⁻ and GFP⁺ cells between treatments, we were unable to guarantee that the data was normally distributed using a two-tailed Kolmogorov-Smirnov test. We therefore used a two-tailed Wilcoxon U-test as it is non-parametric. We also performed two-tailed Student's t-tests, which were in perfect agreement with the results from the U-tests. The same was true when comparing length of the repeat tracts in clones sorted from different populations. All statistical analyses were done using R Studio version 0.99.441. We concluded that a significant difference existed when P<0.05.

Results

A GFP-based assay to detect both expansions and contractions of CAG repeats

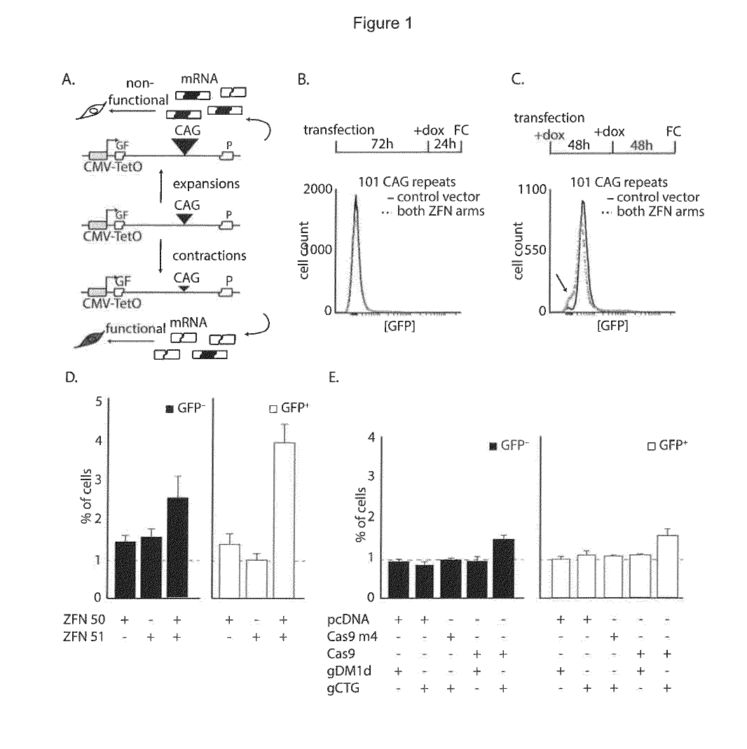

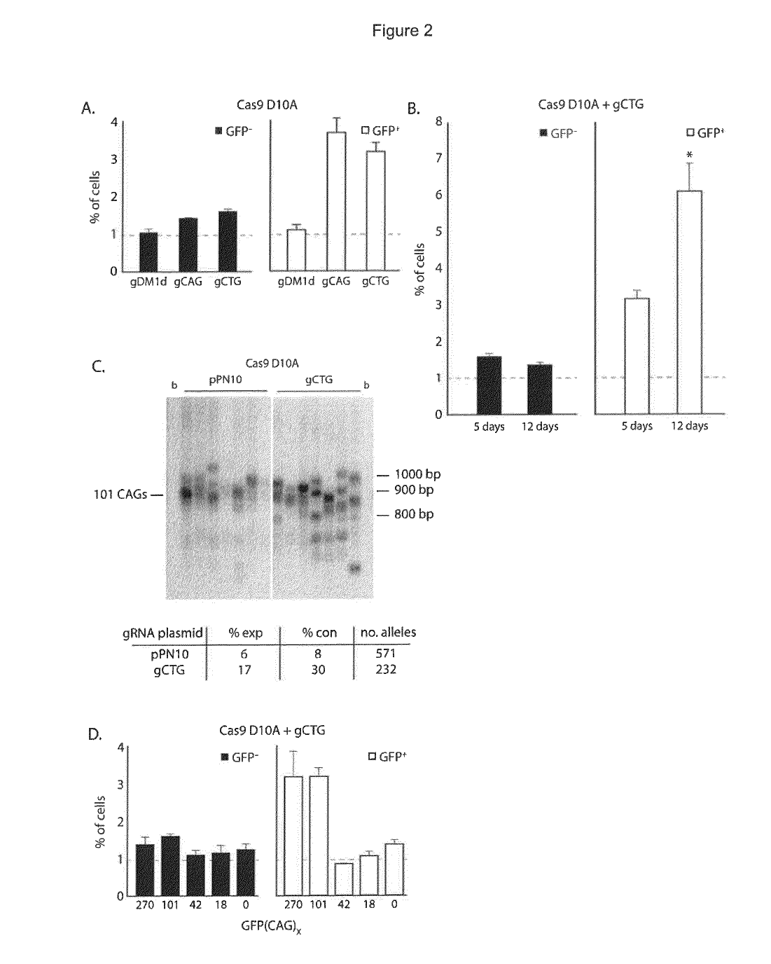

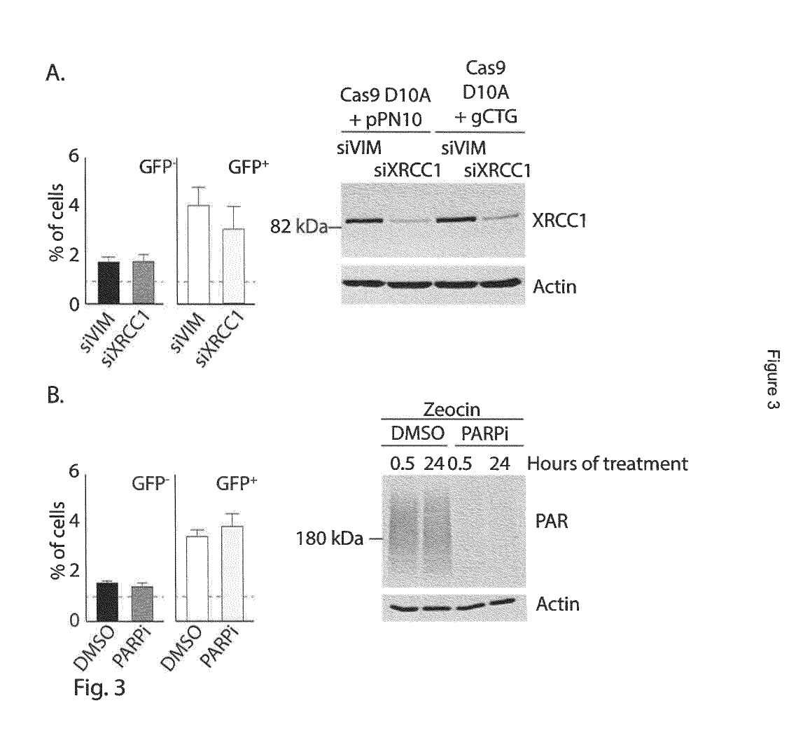

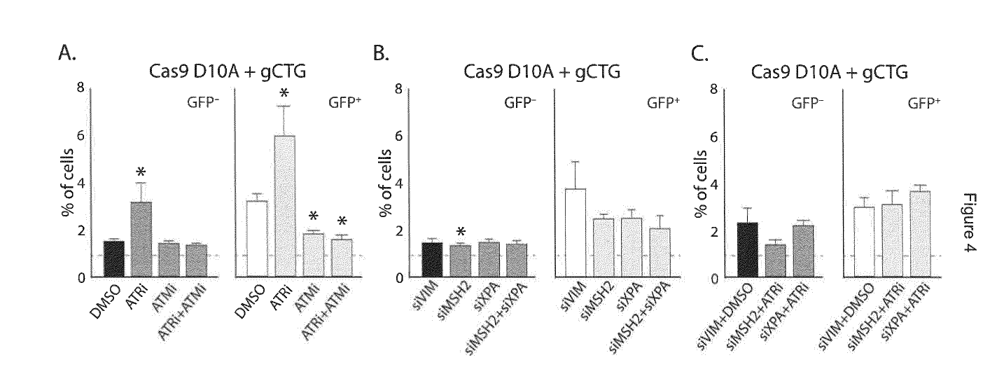

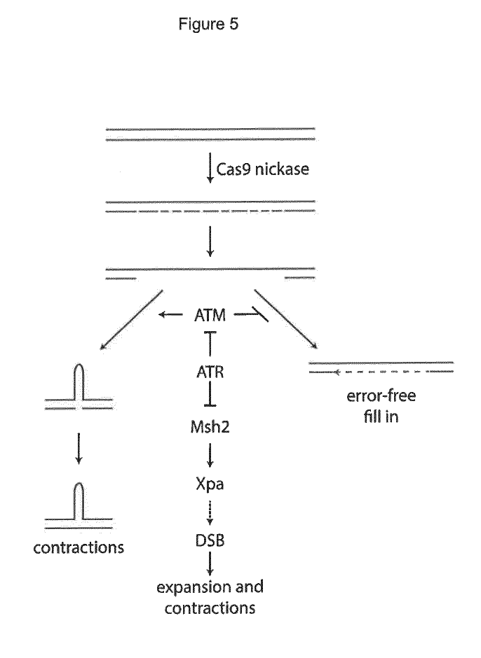

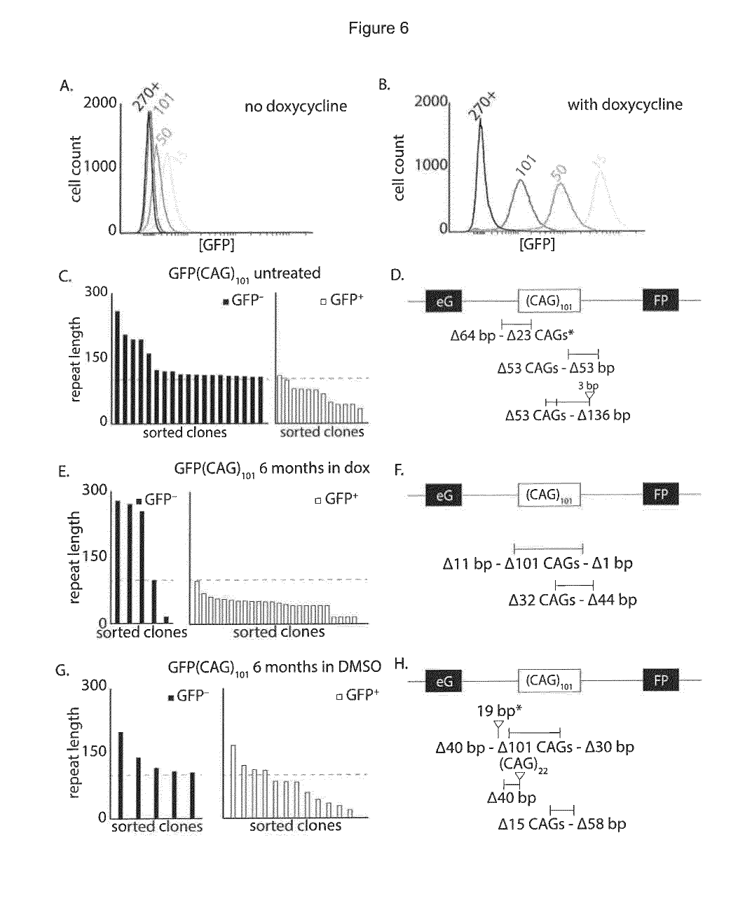

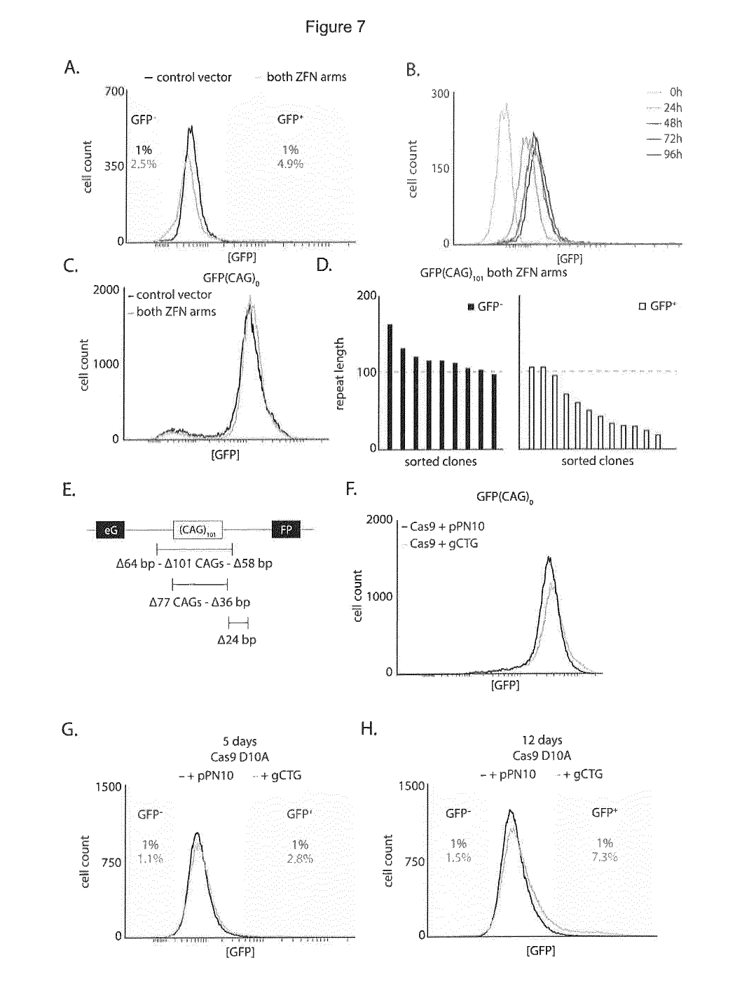

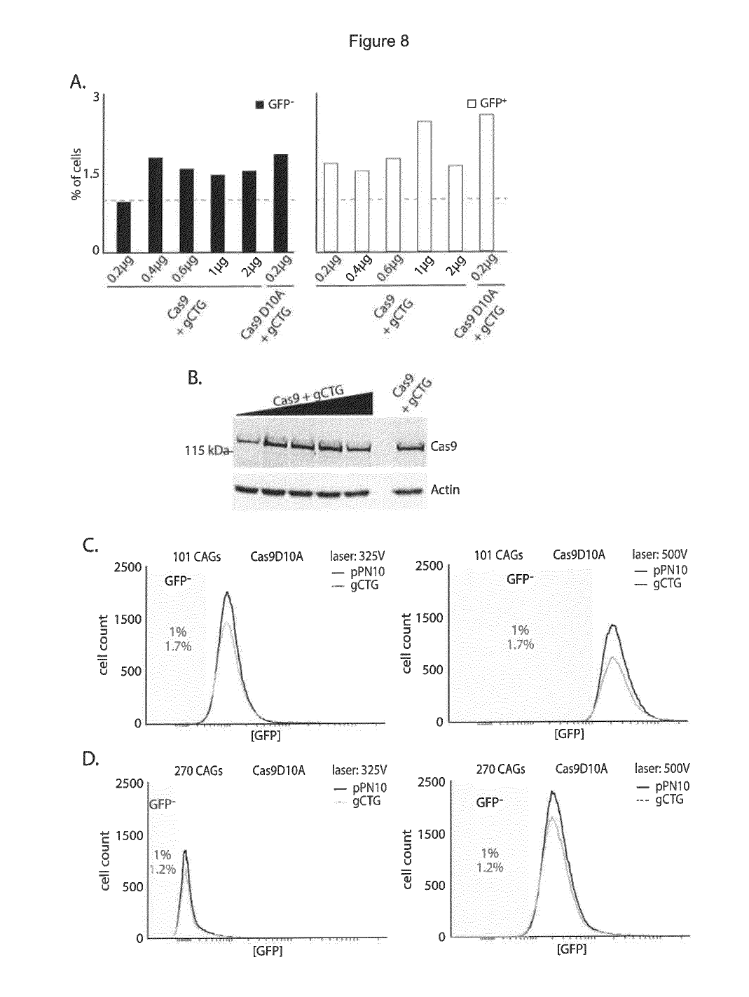

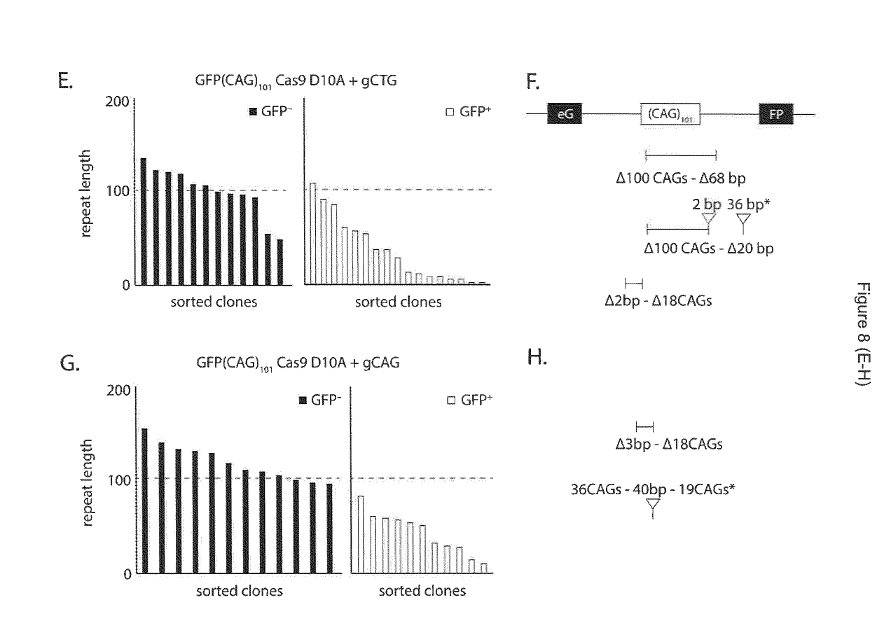

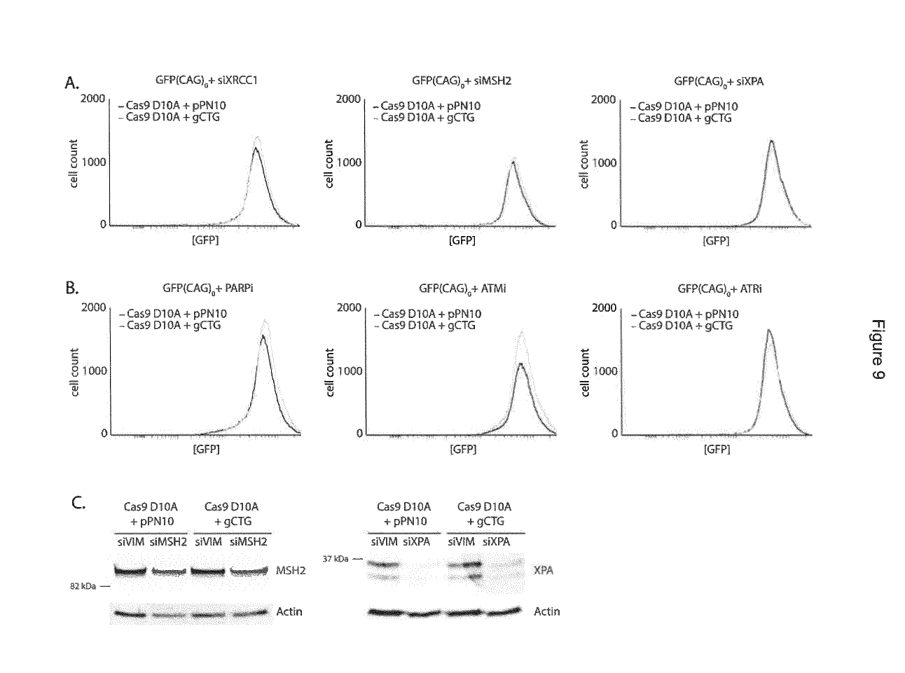

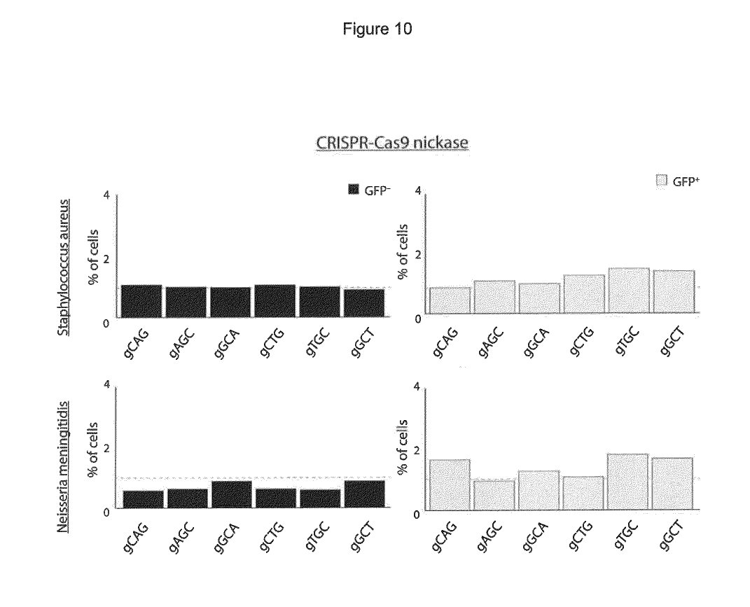

We made use of a recently described GFP-based assay capable of detecting contractions in human cells³ (FIG. 1A). In this assay, CAG repeats within the intron of a GFP mini-gene interfere with splicing in a repeat length-dependent manner, with longer repeats diminishing GFP production. Thus, GFP intensities, measured by flow cytometry, serve as a proxy for the length of the repeat tract (FIG. 6AB). The reporter is present as a single copy integrated in the genome of human HEK293 T-Rex Flp-In cells. It is driven by a doxycycline (dox)-inducible promoter. A second isogenic cell line, GFP(CAG)₀, harbours the same reporter at the same genomic location but it is devoid of CAG repeats. Santillan et al³ validated the assay by expressing a ZFN that cuts the CAG repeat tract. This treatment increased the number of GFP⁺ cells by about 3.5-fold, suggestive of the presence of contractions. They did not report an effect on expansion.

To determine whether we could monitor expansions using by this assay, we sorted GFP⁺ and GFP⁻ cells from a cell population with an average repeat length of 101 CAGs within the GFP reporter (GFP(CAG)₁₀₁) using fluorescence assisted cell sorting (FACS). We defined GFP⁻ cells as those that express GFP at an intensity lower or equal to the bottom 1% of all cells in the population. Similarly, GFP⁺ cells are those expressing at least as much GFP as the brightest 1% of the cells. From the GFP⁻ population, we isolated 19 clones with expansions reaching up to 258 CAGs (FIG. 6C). Of the 12 GFP⁺ clones, 11 had contractions down to 33 CAGs. Sequencing the region flanking the CAG repeats also uncovered deletions in 5 single clones with contractions (FIG. 6D). With the exception of one clone that contained a complex rearrangement, the clones with deletions included 2 bp of microhomology at the junction, suggesting that a minor CAG repeat instability pathway is due to an error-prone alternative end-joining mechanism. Similar results were obtained after FACS of cells from populations that were kept in culture for 6 months with or without dox (FIG. 6E-H). These results demonstrate that the assay can detect expansions that nearly triple the size of the repeat tract.

Double-strand breaks induce both contractions and expansions

To determine whether ZFN-induced expansions as well as the contractions reported by Santillan et al, we repeated the same experiment³. Here we defined GFP⁻ and GFP⁺ cells as those containing GFP intensities in the brightest and dimmest 1% after transfection with the control vector, pCDNA3.1 (FIG. 7A). We reproduced their results: ZFN expression increased the frequency of GFP⁺ cells by 3.2 fold, but had no effect on the number of GFP⁻ cells (FIG. 1B). While optimizing the assay, we noted that GFP intensities increased upon the addition of dox for 72 hours before reaching a steady-state level (FIG. 7B). This is in contrast to the 24 hours previously reported³. Increasing the time of GFP induction raised the overall apparent average intensity of GFP and unmasked an additional GFP⁻ cell population only in the sample transfected with both ZFN arms (FIG. 1C—arrow). This approach revealed 2.5 and 3.9 -fold increases in the proportion of GFP⁻ and GFP⁺ cells, respectively, upon expression of both ZFN arms compared to transfecting an empty control (FIG. 1D).

Expressing either one ZFN arm led to small increases between 1.4 and 1.5 for GFP—cells and between 0.98 and 1.4 for GFP+ cells (FIG. 1D). Expressing both ZFN arms in the GFP(CAG)₀ cell line had no effect on GFP intensities (FIG. 7C). We confirmed that GFP⁻ cells contained expansions and GFP⁺ cells harbored contractions by sorting cells exposed to both ZFN arms. Of the 9 GFP⁻ clones analyzed, 8 revealed an expansion (FIG. 7DE). None of them contained deletions and were therefore not GFP⁻ because they had lost the GFP reporter. Of the 13 GFP⁺ clones, 11 had contractions. Of those, 3 had deletions in the flanking sequences, which is similar to the findings of a previous study constrained to measuring only contractions and using a different ZFN. These results demonstrate that GFP⁻ and GFP⁺ cells accurately reflect the presence of expansions and contractions, making this assay especially well-suited to detect quickly expansions and contractions within a chromosomal environment.

To confirm that DSBs within the repeat tract lead to both expansions and contractions, we used a second type of programmable nuclease: CRISPR-Cas9. This bacterial nuclease is guided to virtually any sequence of interest by a guide RNA (gRNA) molecule where it induces blunt-ended DSBs, making it a highly effective gene editing tool⁶. Expressing Cas9 together with a gRNA composed of a CTG repeat (gCTG) resulted in a meek 1.4 and 1.5 fold induction of GFP⁻ and GFP⁺ cells, respectively, compared to Cas9 expression vector co-transfected with the empty gRNA vector (pPN10) (FIG. 1E). This low efficiency may reflect that the protospacer adjacent motif (PAM) next to the target sequence of gCTG is not the ideal NGG. Transfection of a vector expressing a gRNA that targets the unrelated DMPK locus (gDMd) together with Cas9 did not affect GFP expression (FIG. 1E). Similarly, expressing the gCTG alone, the nuclease and gCTG in the GFP(CAG)₀ cell line, or the gCTG together with a nuclease dead version of Cas9 (Cas9m4) did not change GFP expression significantly (FIG. 1E, 7F). We conclude that DSBs induced by a ZFN or the Cas9 nuclease provoke nearly as many expansions as contractions.

The Cas9 nickase induces CAG repeat contractions

The use of the Cas9 enzyme allowed us to test whether the type of DNA damage present within the repeat tract influences CAG repeat instability. Indeed, the Cas9 D10A mutant can be used with the same gRNA to introduce DNA nicks on the strand complementary to the gRNA. DNA nicks are important intermediates in repeat instability in vitro¹. In mammalian cell lines the chemical inhibition or knockdown of SSBR proteins increases contractions, but the effect on expansion was not assayed and remains unknown².

We found that expressing the nickase together with gCTG in GFP(CAG)₁₀₁ cells increased the number of GFP⁻ cells by 1.6-fold and GFP⁺ cells by 3.2-folds compared to cells expressing only the nickase (FIG. 2A). Transfecting Cas9 nickase with gCAG had a similar effect, leading to increases of 1.4 and 3.7 folds in GFP⁻ and GFP⁺ cells, respectively (FIG. 2A). To control for a potential indirect effect on GFP expression, we expressed the Cas9 nickase along with gDMd, which targets the unrelated DMPK. This had no effect on GFP expression (FIG. 2A). In addition, the gCTG alone did not induce GFP⁺ cells, neither did the expression of gCTG together with the Cas9m4 mutant (FIG. 1E), suggesting that the activity of the nickase is necessary. The increase in GFP⁺ cells was dependent on the presence of the repeats since the nickase together with gCTG had no effect in GFP(CAG)₀ (FIG. 8A). In addition, the Cas9 nickase did not increase the number of dead cells, which could skew the quantification of GFP⁺ and GFP⁻ cells (Table 7). The difference in the number of GFP⁺ cells induced between the nuclease and the nickase was not due to differences in expression levels of the Cas9 enzyme (FIG. 8BC). This suggests that Cas9-nickase leads to instability with a bias towards contractions.

TABLE 7
Cell viability

| Treatment | | Viability %* |
|---|---|---|
| pcDNA 3.1 Zeo | | 76.6 |
| ZFN 50 | | 81.6 |
| ZFN 51 | | 79.8 |
| ZFNs | | 75 |
| Cas9 + pPN10 | | 76.9 |
| Cas9 + gDMd | | 76.1 |
| Cas9 + gCTG | | 85.4 |
| Cas9 D10A + pPN10 | | 77.3 |
| Cas9 D10A + gDMd | | 75.8 |
| Cas9 D10A + gCTG | DMSO | 81 |
| | ATRi | 82.6 |
| | ATMi | 77.2 |
| | PARPi | 75.9 |

*derived from three experiments.

We further confirmed these results using small-pool PCR directly after sorting nickase and gCTG transfected GFP(CAG)₁₀₁ cells (FIG. 2C). Indeed, GFP⁺ cells carried large contractions not seen frequently in cells expressing GFP intensities in the 25th to 75th percentiles. However, the GFP⁻ cell population showed a large variation in repeat length, suggesting that there was no large increase in the number of expansions in this population. In addition, we treated cells for 12 days with the Cas9 nickase. This induced GFP⁺ by 5.8 fold and GFP⁻ by only 1.3 fold in the chromosomal reporter. We further tested whether the way we quantified the data induced a bias against expansions (FIG. 8CD). This was not the case. We conclude that the Cas9 nickase targeted by gCAG or gCTG leads to expansions only rarely. The large bias towards contractions is in sharp contrast to the results we obtained with the ZFNs and the Cas9 nuclease.

We next examined the effect of repeat length on Cas9 nickase-induced contractions. To do so, we used GFP(CAG)ₓ cell lines with repeat sizes ranging from 0 to 270. We detected no substantial increase in GFP⁻ cells upon expression of both the Cas9 nickase and gCTG as the size of the repeat tract increased (FIG. 2D). By contrast, the same treatment increased the proportion of GFP⁺ cells in GFP(CAG)₂₇₀ and GFP(CAG)₁₀₁ cells, but not in GFP(CAG)₄₂, GFP(CAG)₁₈, nor GFP(CAG)₀ (FIG. 2D). These observations suggest that normal-length repeats are not prone to contractions upon expression of the Cas9-nickase. We further substantiated this claim by examining the extent of the Cas9-induced changes at repeats of normal sizes at seven different CAG-repeat containing loci in the genome (Table 8). We used 9 GFP⁺ clones that had a contracted CAG repeat at the GFP reporter due to the action of the Cas9 nickase guided by gCTG. We found that the 126 alleles sequenced remained mutation-free (Table 9), suggesting that the off-target effect of this treatment is negligible.

TABLE 8
Sequences of loci with
CAG/CTG repeats in GFP(CAG)₁₀₁

| Locus | Sequence | SEQ IDs |
|---|---|---|
| AR | (CAG)₂₀-CAA GAG ACT AGC | SEQ ID No. 24 |
| | CCC AGG (CAG)₅ | |
| AR | (CAG)₂₁-CAA GAG ACT AGC | SEQ ID No. 25 |
| | CCC AGG (CAG)₅ | |
| ATN1 | CAG-CAA-CAG-CAA-(CAG)₁₅ | SEQ ID No. 26 |
| ATN1 | CAG-CAA-CAG-CAA-(CAG)₁₆ | SEQ ID No. 27 |
| ATXN1 | (CAG)₁₂-CAT-CAG-CAT-(CAG)₁₁ | SEQ ID No. 28 |
| ATXN1 | (CAG)₁₂-CAT-CAG-CAT-(CAG)₁₂ | SEQ ID No. 29 |
| DMPK | (CTG)₅ | SEQ ID No. 30 |
| PPP2R2B | (CAG)₁₀ | SEQ ID No. 31 |
| TBP | (CAG)₃-(CAA)₃-(CAG)₉-CAA- | SEQ ID No. 32 |
| | CAG-CAA-(CAG)₁₈-CAA-CAG | |
| TBP | (CAG)₃-(CAA)₃-(CAG)₉-CAA- | SEQ ID No. 33 |
| | CAG-CAA-(CAG)₁₉-CAA-CAG | |
| TCF4 | (CTG)₁₄-(CTC)₆ | SEQ ID No. 34 |
| TCF4 | (CTG)₁₅-(CTC)₆ | SEQ ID No. 35 |
| TCF4 | (CTG)₁₆-(CTC)₆ | SEQ ID No. 36 |
| TCF4 | (CTG)₁₇-(CTC)₆ | SEQ ID No. 37 |

TABLE 9
Effect of the Cas9 nickase targeted by gCTG
at other CAG/CTG sites in the genome.

| | | |
|---|---|---|
| | no. of | |
| no. of repeats* | alleles | no. with |

| Locus | Allele 1 | Allele 2 | sequenced | changes |
|---|---|---|---|---|
| AR | 20 + 5 | 21 + 5 | 18 | 0 |
| ATN1 | 15 | 16 | 18 | 0 |
| ATXN1 | 12 + 11 | 12 + 12 | 18 | 0 |
| DMPK | 5 | 5 | 18 | 0 |
| PPP2R2B | 10 | 10 | 18 | 0 |
| TBP | 9 + 18 | 9 + 19 | 18 | 0 |
| TCF4 | 14 | 17 | 18 | 0 |

SSBR is not involved in Cas9 nickase-induced contractions

Having determined that the Cas9 nickase induced CAG repeat instability with a large bias towards contractions without detectable off-target mutations, we next sought to define the mutagenic intermediate that leads to contractions. The experiments with the ZFN and the Cas9 nuclease suggest that the mechanism of nickase-induced repeat contraction probably does not involve DSB repair since that would lead to both expansions and contractions (FIGS. 1, 6, 7). Moreover, Wilson and colleagues have shown that the expression of a dominant negative mutant of Rad51 reduced the frequencies of CAG repeat contractions in a HPRT-based assay, which is blind to expansions¹⁴.

We considered that the Cas9 nickase could induce either nicks sparsely along the repeat tract that would be substrates for SSB repair, or could generate a high density of single-strand breaks within the repeat and create a DNA gap. If nicks were the mutagenic intermediates, then inhibiting SSB repair should further increase the number of GFP⁺ cells after co-expression of the nickase and the gCTG. We therefore interfered with the SSB repair pathway in two different ways: by knocking down XRCC1 and by inhibiting PARP with Oliparib. Both factors are essential to recruit the DNA ligase and efficiently repair SSBs. Neither treatment changed the frequency of GFP⁺ cells compared to controls (FIG. 3AB). This is despite the XRCC1 protein levels being substantially reduced and Olipabib leading to an accumulation of G2 cells and inhibiting PARylation in response to zeocin treatment (Table 10, FIG. 3AB). These observations suggest that isolated nicks do not provoke repeat contractions. More likely, the mutagenic intermediate is a DNA gap created by several closely spaced nicks.

TABLE 10
Cell cycle analysis upon inhibitor treatment

| Treatment | Inhibitor | >2n | G1 | S | G2 | >4n |
|---|---|---|---|---|---|---|
| Cas9 | DMS | 4.3± | 50.0± | 18.8± | 20.2± | 6.2± |
| D10A + | ATMi | 7.5± | 34.9± | 15.3± | 37.2± | 4.9 ± 1 |
| gCTG | ATRi | 2.0± | 41.4± | 20.9± | 25.4± | 10.3 ± 3 |
| | PARPi | 5.0± | 40.7± | 19.0± | 30.0± | 5.3 ± 1 |

* n = 4 for each treatment. Average % of cells ± standard deviation.

ATR and ATM in Cas9 nickase-induced CAG repeat instability

UV-induced DNA gaps activate ATR. We therefore tested the effect of inhibiting this DDR kinase using the small molecule VE-821. We found that VE-821 treatment led to a 3.1- and 5.9-fold increase in GFP⁻ and GFP⁺ cells, respectively, when used in combination with the Cas9-nickase and gCTG (P=0.03 compared to DMSO-treated cells) (FIG. A). This simultaneous treatment did not affect GFP expression in GFP(CAG)₀ (FIG. 9B), confirming that the effect depends on Cas9 activity within the expanded repeat tract. By contrast, using KU60019 to inhibit ATM, which has overlapping as well as distinct roles compared to ATR in the DDR, led to a nearly two-fold reduction in the frequency of GFP⁺ cells (P=0.6 compared to ATMi treatment alone). Intriguingly, treating cells simultaneously with both ATM and ATR inhibitors reduced the number of contractions induced by the Cas9 nickase, similar to using the ATM inhibitor alone (P=0.03 compared to ATM treatment alone). These results argue that ATR prevents CAG repeat instability upon Cas9-nickase activity and works upstream of ATM, which promotes the formation of contractions.

Cas9 nickase-induced GFP⁺ cells are independent of MSH2 and XPA

We next aimed to further define how the Cas9 nickase leads to a contraction bias. We first considered the role of the mismatch repair protein MSH2, because its role in repeat instability during mouse spermatogenesis was proposed to occur through a DNA gap intermediate. We found that MSH2 knockdown did not consistently reduce the number of Cas9 nickase-induced GFP⁺ cells compared to a control knockdown of vimentin (FIG. 4B P=0.24). MSH2 promotes CAG repeat contractions together with the transcription-coupled NER. It was therefore not surprising that the knockdown of XPA, a key component of NER, did not significantly reduce the frequency of nickase-induced GFP⁺ cells, and that neither did the simultaneous knockdown of MSH2 and XPA (FIG. 4B, P=0.24 for XPA, P=0.31 for XPA-MSH2). These results argue that neither MSH2 nor XPA are involved in generating contractions at Cas9 nickase-induced lesions.

We reasoned that ATR inhibition may be increasing the number of expansions and contractions because of double-strand break intermediates that form under these conditions. We therefore tested whether the NER pathway, which is known to generate DSBs upon UV damage and at short inverted repeats, could contribute to repeat instability in the absence of ATR activity. Knockdown of XPA in cells treated with VE-821 led to results indistinguishable from those obtained when treating cells with DMSO together with a control siRNA (P=0.18, FIG. 4C). Similarly, the effect of VE-821 treatment was suppressed by MSH2 knockdown (P=0.85 compared to control DMSO and vimentin siRNA treatment, FIG. 4C), as is expected if MSH2 and XPA work together at expanded repeat tracts. These results suggest that expansions and contractions induced by the inhibition of ATR occur because of a XPA- and MSH2-dependent generation of DSBs.

### Example 2

Use of Cas9 nickase orthologues to induce contractions.

We have expressed Cas9 nickases from N. meningitidis and S. aureus. Both induced more contractions than expansions, provided the correct sgRNA, in GFP(CAG)₁₀₁ cells is used.

### REFERENCES

1. Lopez Castel, A., Cleary, J. D. & Pearson, C. E. Repeat instability as the basis for human diseases and as a potential target for therapy. Nat Rev Mol Cell Biol 11, 165-70 (2010).
2. Usdin, K., House, N. C. & Freudenreich, C. H. Repeat instability during DNA repair: Insights from model systems. Crit Rev Biochem Mol Biol 50, 142-67 (2015).
3. Santillan, B. A., Moye, C., Mittelman, D. & Wilson, J. H. GFP-based fluorescence assay for CAG repeat instability in cultured human cells. PLoS One 9, e113952 (2014).
4. Mahadevan, M. S. et al. Reversible model of RNA toxicity and cardiac conduction defects in myotonic dystrophy. Nat Genet 38, 1066-70 (2006).
5. Zu, T. et al. Recovery from polyglutamine-induced neurodegeneration in conditional SCA1 transgenic mice. J Neurosci 24, 8853-61 (2004).
6. Sternberg, S. H. & Doudna, J. A. Expanding the Biologist's Toolkit with CRISPR-Cas9. Mol Cell 58, 568-74 (2015).
7. Tsai, S. Q. et al. GUIDE-seq enables genome-wide profiling of off-target cleavage by CRISPR-Cas nucleases. Nat Biotechnol 33, 187-97 (2015).
   8. Esvelt, K. M. et al. Orthogonal Cas9 proteins for RNA-guided gene regulation and editing. Nat Methods 10, 1116-21 (2013).
   9. Lahiri, M., Gustafson, T. L., Majors, E. R. & Freudenreich, C. H. Expanded CAG repeats activate the DNA damage checkpoint pathway. Mol Cell 15, 287-93 (2004).
   10. Entezam, A. & Usdin, K. ATR protects the genome against CGG.CCG-repeat expansion in Fragile X premutation mice. Nucleic Acids Res 36, 1050-6 (2008).
   11. Entezam, A. & Usdin, K. ATM and ATR protect the genome against two different types of tandem repeat instability in Fragile X premutation mice. Nucleic Acids Res 37, 6371-7 (2009).
   12. Wheeler, V. C. et al. Mismatch repair gene Msh2 modifies the timing of early disease in Hdh(Q111) striatum. Hum Mol Genet 12, 273-81 (2003).
   13. Dion, V., Lin, Y., Hubert, L., Jr., Waterland, R. A. & Wilson, J. H. Dnmtl deficiency promotes CAG repeat expansion in the mouse germline. Hum Mol Genet 17, 1306-17 (2008).
   14. Mittelman, D. et al. Zinc-finger directed double-strand breaks within CAG repeat tracts promote repeat instability in human cells. Proc Natl Acad Sci USA 106, 9607-12 (2009).
   15. Naldini et al., Lentiviral vectors, two decades later. Science. 2016 Sept. 9; 353 (6304):1101-2
   16. Dull T, Zufferey R, Kelly M, Mandel R J, Nguyen M, Trono D, Naldini L. J Virol. 1998 November; 72(11):8463-71
   17. Ran et al., Genome engineering using the CRISPR-Cas9 system. Nature Protocols 8, 2281-2308 (2013)

1. A kit for the treatment and/or prevention of a DNA-triplet repeat disease comprising a gene delivery vector, said vector comprising
i) a Cas9 nickase optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

2. The kit for the treatment and/or prevention of a DNA-triplet repeat disease of claim 1, characterized in that said DNA-triplet repeat disease is an expanded CAG/CTG repeat disorder.

3. The kit for the treatment and/or prevention of a DNA-triplet repeat disease of claim 2, characterized in that said DNA-triplet repeat disease is a neurological disease or a neuromuscular disease.

4. The kit for the treatment and/or prevention of a DNA-triplet repeat disease of claim 3, characterized in that said neurological disease or neuromuscular disease is selected from the group comprising Dentatorubral-pallidoluysian atrophy, Fuchs' endothelial corneal dystrophy, Huntington disease, Huntington disease-Like 2. Myotonic Dystrophy type 1, Spinal and bulbar muscular atrophy, spinocerebellar ataxia 1, spinocerebellar ataxia 2, spinocerebellar ataxia 3, spinocerebellar ataxia 6, spinocerebellar ataxia 7, spinocerebellar ataxia 8, spinocerebellar ataxia 12, and spinocerebellar ataxia 17.

5. The kit for the treatment and/or prevention of a DNA-triplet repeat disease of anyone of the preceding claims, characterized in that the target sequence comprising 16 to 25 nucleotides is selected from the group comprising CAG CAG CAG CAG CAG CAG CAG (SEQ ID No. 1) and CTG CTG CTG CTG CTG CTG CTG (SEQ ID No. 2), or fragments thereof.

6. The kit for the treatment and/or prevention of a DNA-triplet repeat disease of anyone of the preceding claims, characterized in that the gene delivery vector is selected from the group comprising an adeno-associated virus (AAV) and a lentivirus.

7. A kit for the treatment and/or prevention of a DNA-triplet repeat disease comprising
i) a first gene delivery vector comprising a Cas9 nickase optimized for gene editing in mammalian cultured cell lines, embryonic stem (ES) cells, induced pluripotent stem cells (iPSCs), or in vivo, and
ii) a second gene delivery vector comprising at least one single guide RNA (sgRNA), or crRNA and tracrRNA, recognizing a target sequence comprising 16 to 25 nucleotide wherein said target sequence is present immediately upstream of a protospacer adjacent motif (PAM).

8. The kit for the treatment and/or prevention of a DNA-triplet repeat disease of claim 7, characterized in that said DNA-triplet repeat disease is an expanded CAG/CTG repeat disorder.

9. The kit for the treatment and/or prevention of a DNA-triplet repeat disease of claim 8, characterized in that said DNA-triplet repeat disease is a neurological disease or a neuromuscular disease.

10. The kit for the treatment and/or prevention of a DNA-triplet repeat disease of claim 9, characterized in that said neurological disease or neuromuscular disease is selected from the group comprising Dentatorubral-pallidoluysian atrophy, Fuchs' endothelial corneal dystrophy, Huntington disease, Huntington disease-Like 2. Myotonic Dystrophy type 1, Spinal and bulbar muscular atrophy, spinocerebellar ataxia 1, spinocerebellar ataxia 2, spinocerebellar ataxia 3, spinocerebellar ataxia 6, spinocerebellar ataxia 7, spinocerebellar ataxia 8, spinocerebellar ataxia 12, and spinocerebellar ataxia 17.

11. The kit for the treatment and/or prevention of a DNA-triplet repeat disease of anyone of claims 7-10, characterized in that the target sequence comprising 16 to 25 nucleotides is selected from the group comprising CAG CAG CAG CAG CAG CAG CAG (SEQ ID No. 1) and CTG CTG CTG CTG CTG CTG CTG (SEQ ID No. 2), or fragments thereof.

12. The kit for the treatment and/or prevention of a DNA-triplet repeat disease of anyone of the preceding claims, characterized in that the first and second gene delivery vectors are independently selected from the group comprising an adeno-associated virus (AAV) and a lentivirus.

13. A gene delivery vector comprising
i) a Cas9 nickase optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

14. A gene delivery vector for use in the treatment and/or prevention of a DNA-triplet repeat disease, said vector comprising
i) a Cas9 nickase optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

15. A pharmaceutical composition comprising
i) a vector comprising a Cas9 nickase optimized for gene editing in mammalian cultured cell lines, embryonic stem (ES) cells, induced pluripotent stem cells (iPSCs), or in vivo, and at least one single guide RNA (sgRNA), or crRNA and tracrRNA, recognizing a target sequence comprising 16 to 25 nucleotides wherein said target sequence is present immediately upstream of a protospacer adjacent motif (PAM), or
ii) a first gene delivery vector comprising an endonuclease Cas9 optimized for gene editing in mammalian cultured cell lines, embryonic stem (ES) cells, induced pluripotent stem cells (iPSCs), or in vivo, and a second gene delivery vector comprising at least one single guide RNA (sgRNA), or crRNA and tracrRNA, recognizing a target sequence comprising 16 to 25 nucleotide wherein said target sequence is present immediately upstream of a protospacer adjacent motif (PAM).

16. Use of a gene delivery vector in the treatment and/or prevention of a DNA-triplet repeat disease, said vector comprising
i) a Cas9 nickase optimized for gene editing in mammalian cultured cell lines, embryonic stem (ES) cells, induced pluripotent stem cells (iPSCs), or in vivo, and
ii) at least one single guide RNA (sgRNA), or crRNA and tracrRNA, recognizing a target sequence comprising 16 to 25 nucleotides wherein said target sequence is present immediately upstream of a protospacer adjacent motif (PAM).

17. A method of treating and/or preventing a DNA-triplet repeat disease comprising administering a pharmaceutical composition of claim 10 to a subject in need thereof.

18. A method of treating and/or preventing a DNA-triplet repeat disease in a subject in need thereof, said method comprising
(a) altering a target sequence in a cell ex vivo by contacting said cell with the gene delivery vector of claim 13, wherein the at least one single guide RNA (sgRNA), or crRNA and tracrRNA, directs Cas9 nickase to and hybridizes to a target motif of the target sequence, thereby cleaving the target sequence,
(b) introducing the cell into the subject, thereby treating and/or preventing said DNA-triplet repeat disease.

19. The method of treating and/or preventing of claim 18, characterized in that the cell is selected from the group comprising a cultured cell line, an embryonic stem (ES) cell, an induced pluripotent stem cell (iPSCs) and a cultured primary cell.

20. The method of treating and/or preventing of claim 18 or 19, characterized in that the cell is selected from the group comprising neurons, glia, satellite muscle cells, heart cells, hepatocytes, and fibroblasts.

21. Use of a pharmaceutical composition of claim 15 in the manufacture of a medicament for the treatment and/or prevention of a DNA-triplet repeat disease.


SEQUENCE LISTING (37 sequences)

SEQ ID NO: 1 - DNA, 21 bp, Unknown
  synthetic sequence
cagcagcagcagcagcagcag

SEQ ID NO: 2 - DNA, 21 bp, Unknown
  synthetic sequence
ctgctgctgctgctgctgctg

SEQ ID NO: 3 - DNA, 114 bp, Unknown
  synthetic sequence
gcagcagcagcagcagcagcaggttgtagctccctttctcatttcggaaacgaaatgagaaccgttgctacaataaggccgtctgaaaagatgtgccgcaacgctctgcccctt

SEQ ID NO: 4 - DNA, 114 bp, Unknown
  synthetic sequence
gctgctgctgctgctgctgctggttgtagctccctttctcatttcggaaacgaaatgagaaccgttgctacaataaggccgtctgaaaagatgtgccgcaacgctctgcccctt

SEQ ID NO: 5 - RNA, 19 nt, Unknown
  synthetic sequence
gaaugguacaaauccaagu

SEQ ID NO: 6 - RNA, 19 nt, Unknown
  synthetic sequence
ucugcagaguguugugcuu

SEQ ID NO: 7 - RNA, 19 nt, Unknown
  synthetic sequence
gcuacuggaggcauggcua

SEQ ID NO: 8 - RNA, 25 nt, Unknown
  artificial sequence
caguuugugaucacagcacaggaau

SEQ ID NO: 9 - DNA, 20 bp, Unknown
  synthetic sequence
taccaggacagcagtggtca

SEQ ID NO: 10 - DNA, 22 bp, Unknown
  synthetic sequence
aagagcttccctttacacaacg

SEQ ID NO: 11 - DNA, 19 bp, Unknown
  synthetic sequence
tctgcaaattcagtgatgc

SEQ ID NO: 12 - DNA, 20 bp, Unknown
  synthetic sequence
gagcgtgggtctccgcccag

SEQ ID NO: 13 - DNA, 20 bp, Unknown
  synthetic sequence
cactttgcgaaccaacgata

SEQ ID NO: 14 - DNA, 20 bp, Unknown
  synthetic sequence
actcagccttctctcccatc

SEQ ID NO: 15 - DNA, 20 bp, Unknown
  synthetic sequence
tgtaggacacctggctgtga

SEQ ID NO: 16 - DNA, 20 bp, Unknown
  synthetic sequence
tagggctgggaagggtctac

SEQ ID NO: 17 - DNA, 20 bp, Unknown
  synthetic sequence
ctctgggacgcaacctctct

SEQ ID NO: 18 - DNA, 20 bp, Unknown
  synthetic sequence
ttccagttcattgggtcctc

SEQ ID NO: 19 - DNA, 20 bp, Unknown
  synthetic sequence
gtgtgtgggatcatcgtctg

SEQ ID NO: 20 - DNA, 20 bp, Unknown
  synthetic sequence
ttctccttgctttccacagg

SEQ ID NO: 21 - DNA, 20 bp, Unknown
  synthetic sequence
ggggagggatacagtggagt

SEQ ID NO: 22 - DNA, 20 bp, Unknown
  synthetic sequence
gcagcaaagagcagccgcag

SEQ ID NO: 23 - DNA, 20 bp, Unknown
  synthetic sequence
ctggtcccacgggagggcgg

SEQ ID NO: 24 - DNA, 93 bp, Unknown
  synthetic sequence
cagcagcagcagcagcagcagcagcagcagcagcagcagcagcagcagcagcagcagcagcaagagactagccccaggcagcagcagcagcag

SEQ ID NO: 25 - DNA, 96 bp, Unknown
  synthetic sequence
cagcagcagcagcagcagcagcagcagcagcagcagcagcagcagcagcagcagcagcagcagcaagagactagccccaggcagcagcagcagcag

SEQ ID NO: 26 - DNA, 57 bp, Unknown
  synthetic sequence
cagcaacagcaacagcagcagcagcagcagcagcagcagcagcagcagcagcagcag

SEQ ID NO: 27 - DNA, 60 bp, Unknown
  synthetic sequence
cagcaacagcaacagcagcagcagcagcagcagcagcagcagcagcagcagcagcagcag

SEQ ID NO: 28 - DNA, 78 bp, Unknown
  synthetic sequence
cagcagcagcagcagcagcagcagcagcagcagcagcatcagcatcagcagcagcagcagcagcagcagcagcagcag

SEQ ID NO: 29 - DNA, 81 bp, Unknown
  synthetic sequence
cagcagcagcagcagcagcagcagcagcagcagcagcatcagcatcagcagcagcagcagcagcagcagcagcagcagcag

SEQ ID NO: 30 - DNA, 15 bp, Unknown
  synthetic sequence
ctgctgctgctgctg

SEQ ID NO: 31 - DNA, 30 bp, Unknown
  synthetic sequence
cagcagcagcagcagcagcagcagcagcag

SEQ ID NO: 32 - DNA, 114 bp, Unknown
  synthetic sequence
cagcagcagcaacaacaacagcagcagcagcagcagcagcagcagcaacagcaacagcagcagcagcagcagcagcagcagcagcagcagcagcagcagcagcagcagcaacag

SEQ ID NO: 33 - DNA, 117 bp, Unknown
  synthetic sequence
cagcagcagcaacaacaacagcagcagcagcagcagcagcagcagcaacagcaacagcagcagcagcagcagcagcagcagcagcagcagcagcagcagcagcagcagcagcaacag

SEQ ID NO: 34 - DNA, 60 bp, Unknown
  synthetic sequence
ctgctgctgctgctgctgctgctgctgctgctgctgctgctgctcctcctcctcctcctc

SEQ ID NO: 35 - DNA, 63 bp, Unknown
  synthetic sequence
ctgctgctgctgctgctgctgctgctgctgctgctgctgctgctgctcctcctcctcctcctc

SEQ ID NO: 36 - DNA, 66 bp, Unknown
  synthetic sequence
ctgctgctgctgctgctgctgctgctgctgctgctgctgctgctgctgctcctcctcctcctcctc

SEQ ID NO: 37 - DNA, 69 bp, Unknown
  synthetic sequence
ctgctgctgctgctgctgctgctgctgctgctgctgctgctgctgctgctgctcctcctcctcctcctc